(19) United States
(12) Patent Application Publication
(10) Pub. No.: US 2015/0344964 A1
(43) Pub. Date: Dec. 3, 2015

(54) PREDICTION OF THE TREATMENT RESPONSE TO AN ANTI-EGFR MOLECULE IN COLORECTAL CANCER PATIENTS

(71) Applicants: MEDIZINISCHE UNIVERSITÄT GRAZ, Graz (AT); I,S.O. ISTITUTO SUPERIORE DI ONCOLOGIA, Genova (IT)

(72) Inventors: Gerald Höfler, Stattegg (AT); Giorgio Stanta, Genova (IT)

(21) Appl. No.: 14/654,721

(22) PCT Filed: Dec. 16, 2013
(86) PCT No.: PCT/EP2013/076772

§ 371 Date: Jun. 22, 2015

(30) Foreign Application Priority Data
Dec. 20, 2012 (GB) 1223025.6

(51) Int. Cl. C12Q 1/68; C07K 16/40; A61K 31/5377; C07K 16/28; A61K 31/517

(52) U.S. Cl. CPC C12Q 1/6886; C07K 16/2863; A61K 31/517; A61K 31/5377; C07K 16/40; A61K 2039/505

(57) ABSTRACT

The present invention relates to a method for predicting the treatment response to an anti-epidermal growth factor receptor (EGFR) molecule in a patient suffering from colorectal cancer. Furthermore, the present invention relates to an anti-EGFR molecule for use in the treatment of a patient suffering from colorectal cancer.

### TECHNICAL FIELD

The present invention relates to a method for predicting the treatment response to an anti-epidermal growth factor receptor (EGFR) molecule in a patient suffering from colorectal cancer. Furthermore, the present invention relates to an anti-EGFR molecule for use in the treatment of a patient suffering from colorectal cancer.

### BACKGROUND OF THE INVENTION

Colorectal cancer (CRC) is the fourth most common cancer in men (after skin, prostate and lung cancer) as well as in women (after skin, breast and lung cancer) (http://www.cancer.gov/cancertopics/wyntk/colon-and-rectal). Approximately 25% of patients diagnosed with CRC already developed metastases; further, a metastatic disease develops in 40 to 50% of newly diagnosed patients (Van Cutsem et al., 2009). Although colorectal carcinomas can metastasize to almost any organ, the liver and the lungs are the most common sites for metastasis (Edge, 2012). Disease relapse after surgery—with or without adjuvant therapy—mostly occurs within three years.

Colorectal cancer chemotherapy is mainly based on the three drugs 5-FU, Oxaliplatin and Irinotecan. The main advance in the management of patients diagnosed with colorectal cancer in the past five years was the development of targeted drugs in addition to the commonly available treatments. Such targeted drugs approved to the market are Cetuximab (also referred to as C225-03, IMC-C225, C225 and ch225), Panitumumab and Bevacizumab, wherein the monoclonal antibodies Cetuximab (Erbitux®) and Panitumumab (Vectibix®) are directed to the epidermal growth factor receptor (EGFR), while the humanized monoclonal antibody Bevacizumab (Avastin®) is directed to all isoforms of the proangiogenic peptide VEGF.

EGFR (also known as HER1 or ERBB1) is a transmembrane glycoprotein tyrosine kinase, which upon activation stimulates various downstream mediators, related to different biological processes such as cell proliferation, angiogenesis, invasion, metastasis and apoptosis. It is often found to be upregulated in cancers and is a key modulator in the process of cell proliferation in both normal and malignant epithelial cells. EGFR plays a critical role in cancer and thus targeting EGFR is considered a promising approach in cancer treatment (Ciaradiello and Tortora, 2001). For this reason, several therapeutic targets (including the above-mentioned antibodies Cetuximab and Panitumumab) have been and are currently developed which are directed to said receptor. Different studies showed that Cetuximab and

Panitumumab are active alone or in therapeutic combination in both, chemorefractory and untreated CRC patients. However, since such biological therapies are relatively expensive and only 10 to 15% of all CRC patients respond to antibody therapy, there is a need for biomarkers predicting the treatment response to anti-EGFR molecules.

Until now, the best predictive biomarker for the efficacy of Cetuximab or Panitumumab is the mutational status of the KRAS gene. With respect to the KRAS gene, it has been reported that patients having somatic activating mutations in said gene do not respond to the anti-EGFR molecules (Van Cutsem, 2009). However, KRAS mutational status alone is not sufficient for predicting the treatment response since although 40% of the CRCs are KRAS mutated, the response rate to the above antibodies is only 10 to 15% (Bardelli & Siena, 2010). Additional factors such as amphiregulin and epiregulin or alterations of downstream effectors of EGFR and KRAS have been proposed in order to explain the unsuccessful treatment with EGFR-targeted antibodies. However, so far none of these factors is presently used in clinical practice, since there is still a need for further studies with respect to these factors (Sartore-Bianchi et al., 2009).

Hence, it would be desirable to identify further biomarkers suitable for predicting the treatment response to anti-EGFR molecules in patients suffering from colorectal cancer.

It has now been found that the response to a treatment with an anti-EGFR molecule may be predicted by performing a single nucleotide polymorphism (SNP) analysis. In particular, it has been found that a known polymorphism located in exon 20 of the EGFR gene and having the number rs1050171 according to the NCBI SNP database (http://www.ncbi.nlm.nih.gov/snp/?term=rs1050171; SEQ ID NO: 1 as depicted in FIG. 6) is a suitable biomarker for predicting the treatment response to anti-EGFR molecules in CRC patients.

Although the above polymorphism is known, until now it has not been described that it can be used for predicting the treatment response of patients suffering from CRC to an anti-EGFR molecule. Some authors have reported a significant association between genotypes AA and AG at rs1050171 and lung cancer, suggesting that individuals carrying these genotypes are more susceptible to lung cancer, independently of their age, smoking status, race, sex and family cancer history (Zhang et al., 2006), while others did not confirm these findings (Choi et al., 2007). Regarding the relationship between this polymorphism and survival, a weak association between genotypes AG and AA and worse outcome in patients with lung cancer under gefitinib therapy has been reported (Sasaki et al., 2008), while no association was detected between genotypes and outcome in patients with Barrett's adenocarcinomas (Marx et al., 2010). Another study reported that such polymorphisms could be used as a prognostic marker in patients with esophageal squamous cell carcinomas, whereby patients harboring genotype GA showed the worst outcome (Kaneko et al., 2010). Furthermore, the polymorphism was identified) in some studies, but no correlations to clinical parameters or patients' outcome were made (Fukushima et al., 2006; Longatto-Filho et al., 2009; Pugh et al., 2007; Taguchi et al., 2008 and Wu et al., 2007).

### OBJECT AND SUMMARY OF THE INVENTION

Therefore, it is an object of the invention to provide a method for predicting the treatment response to an anti-EGFR molecule in patients suffering from colorectal cancer and to identify patients who are likely to respond to a treatment with anti-EGFR molecules.

In one aspect, the present invention thus relates to a method for predicting the treatment response to an anti-EGFR molecule in a patient suffering from colorectal cancer, comprising

a) providing a nucleic acid sample from the patient suffering from colorectal cancer,

b) performing a single nucleotide polymorphism (SNP) genotyping analysis at rs1050171 on said sample, wherein genotype GG at rs1050171 is indicative for a positive treatment response to an anti-EGFR molecule.

In one of its embodiments the method described herein further comprises a step c) of determining the EGFR expression level if the SNP genotyping analysis shows genotype AG or AA at rs1050171, wherein genotypes AG or AA at rs1050171 in combination with a high EGFR expression level are indicative for a positive treatment response to an anti-EGFR molecule.

In another embodiment the method described herein further comprises a step d) of determining the KRAS mutational status, wherein genotypes AG or AA at rs1050171 in combination with a high EGFR expression level and wild-type KRAS status are indicative for a positive treatment response to an anti-EGFR molecule.

In a further embodiment of the method described herein, the anti-EGFR molecule for which a treatment response is to be predicted is selected from the group consisting of anti-EGFR antibodies, small molecules directed to EGFR and inhibitory polynucleotides capable of interfering with the expression and/or function of EGFR.

In one embodiment of the method described herein, the anti-EGFR molecule for which a treatment response is to be predicted is an anti-EGFR antibody.

One embodiment of the present invention relates to the method described herein, wherein the anti-EGFR antibody for which a treatment response is to be predicted is selected from the group consisting of Cetuximab and Panitumumab.

In another embodiment of the method described herein, the anti-EGFR molecule for which a treatment response is to be predicted is a small molecule directed to EGFR.

In a further embodiment of the method described herein, the small molecule directed to EGFR for which a treatment response is to be predicted is selected from the group consisting of Erlotinib and Gefitinib.

One embodiment of the invention relates to a method as described herein, wherein the patient suffering from colorectal cancer is a patient suffering from metastatic colorectal cancer.

A further aspect of the invention relates to an anti-EGFR molecule for use in the treatment of a patient suffering from colorectal cancer, wherein the patient exhibits

a) genotype GG at rs1050171 or

b) genotype AG or AA at rs1050171 and a high expression level of EGFR.

The expression level of EGFR preferably relates to the mRNA or protein expression level of EGFR, wherein the EGFR mRNA expression level can be particularly preferred.

In one embodiment, the patient as defined under item b) exhibits a wild-type KRAS status.

In another embodiment, the anti-EGFR molecule for use in the treatment of a patient suffering from colorectal cancer is selected from the group consisting of anti-EGFR antibodies, small molecules directed to EGFR and inhibitory polynucleotides capable of interfering with the expression and/or function of EGFR.

In a further embodiment, the anti-EGFR molecule for use in the treatment of a patient suffering from colorectal cancer is an anti-EGFR antibody.

In another embodiment, the anti-EGFR antibody for use in the treatment of a patient suffering from colorectal cancer is selected from the group consisting of Cetuximab and Panitumumab.

In a further embodiment, the anti-EGFR molecule for use in the treatment of a patient suffering from colorectal cancer is a small molecule directed to EGFR.

According to another embodiment, the small molecule directed to EGFR is selected from the group consisting of Erlotinib and Gefitinib.

In another embodiment, the anti-EGFR molecule is for use in the treatment of a patient suffering from metastatic colorectal cancer.

Another aspect of the present invention relates to a kit or diagnostic composition for the analysis of rs1050171 as single nucleotide polymorphism indicative for the treatment response to an anti-EGFR molecule, comprising at least one primer and/or probe for determining the genotype at rs 1050171.

### BRIEF DESCRIPTION OF THE DRAWINGS

FIG. 1 depicts Kaplan-Meier curves showing progression free survival in FIG. 1a and overall survival in FIG. 1b, in patients with a wild type or a mutated KRAS colorectal tumor.

FIG. 2 depicts Kaplan-Meier survival curves showing progression free survival (PFS, FIGS. 2a and 2b) and overall survival (OS; FIGS. 2c and 2d) in colorectal cancer patients according to the different alteration types of the biomarker. In FIG. 2a and FIG. 2c Kaplan-Meier survival curves comparing all three alteration types are reported. In FIG. 2b and FIG. 2d alteration types 2 and 3 are joined and compared to alteration type 1 (TYPE1=genotype GG, TYPE2=genotype AG, TYPE3=genotype AA).

FIG. 3 depicts Kaplan-Meier curves showing overall survival in patients without biological therapy. In FIG. 3a the effect on survival of the three alteration types is shown, while in FIG. 3b Kaplan-Meier survival curve comparing alteration type 1 to joint alteration types 2 and 3 is reported (TYPE1=genotype GG, TYPE2=genotype AG, TYPE3=genotype AA).

FIG. 4 depicts Kaplan-Meier survival curves showing progression free survival (PFS) in FIG. 4a and OS in FIG. 4b in colorectal cancer patients according to the mRNA expression levels of EGFR.

FIG. 5 depicts Kaplan-Meier curves showing progression free survival in patients with a wild-type KRAS in FIG. 5a and in those with a mutated KRAS in FIG. 5b, according to mRNA expression levels of EGFR.

FIG. 6 depicts the sequence of rs1050171 according to the NCBI SNP database.

FIGS. 7 to 9 depict exemplary KRAS, PIK3CA and BRAF mutations, respectively as published by De Roock et al. (2010 b).

Relative mutation distribution=percentage of specific mutation within the mutant subpopulation.

Absolute mutation frequency=percentage of specific mutations in the whole studied population=incidence.

# assays for hotspot mutations that needed to succeed and not show a mutation for a sample to be called wild-type

$ KRAS mutations, PIK3CA mutations, BRAF mutations respectively detected in 6183/17316, 527/3561, 2463/21950 colorectal adenocarcinomas in the COSMIC database.

* including five PIK3CA double mutants and four KRAS double mutants

° total incidence of PIK3CA/KRAS mutant tumours (double mutants counted as one mutant)

§ KRAS, PIK3CA, BRAF and NRAS mutation status missing in 26/773, 30/773, 12/773 and 129/773 respectively

### DETAILED DESCRIPTION OF THE INVENTION

The inventors of the present application inter alia surprisingly found that rs1050171 may be used as a marker for predicting the treatment response to an anti-EGFR molecule in patients suffering from colorectal cancer.

Where the term “comprise” or “comprising” is used in the present description and claims, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

Where an indefinite or a definite article is used when referring to a singular noun e.g. “a” or “an”, “the”, this includes a plural form of that noun unless specifically stated. Vice versa, when the plural form of a noun is used it refers also to the singular form. For example, when anti-EGFR molecules are mentioned, this is also to be understood as a single anti-EGFR molecule or a anti-EGFR molecule of a single type.

Furthermore, the terms first, second, third or (a), (b), (c) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%. The aforementioned deviation from the indicated numerical interval of ±10%, and preferably of ±5% is also indicated by the terms “about” and “approximately” used herein with respect to a numerical value.

As has been discussed above, there is a need for biomarkers allowing to predict the treatment response of patients suffering from colorectal cancer (CRC) to anti-EGFR molecules.

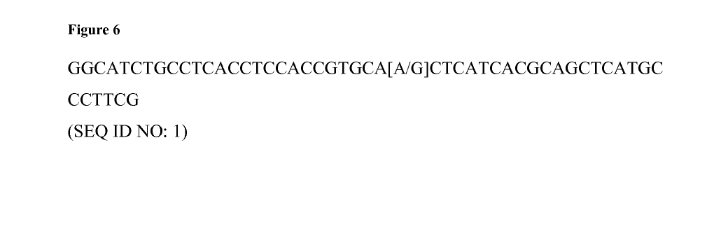

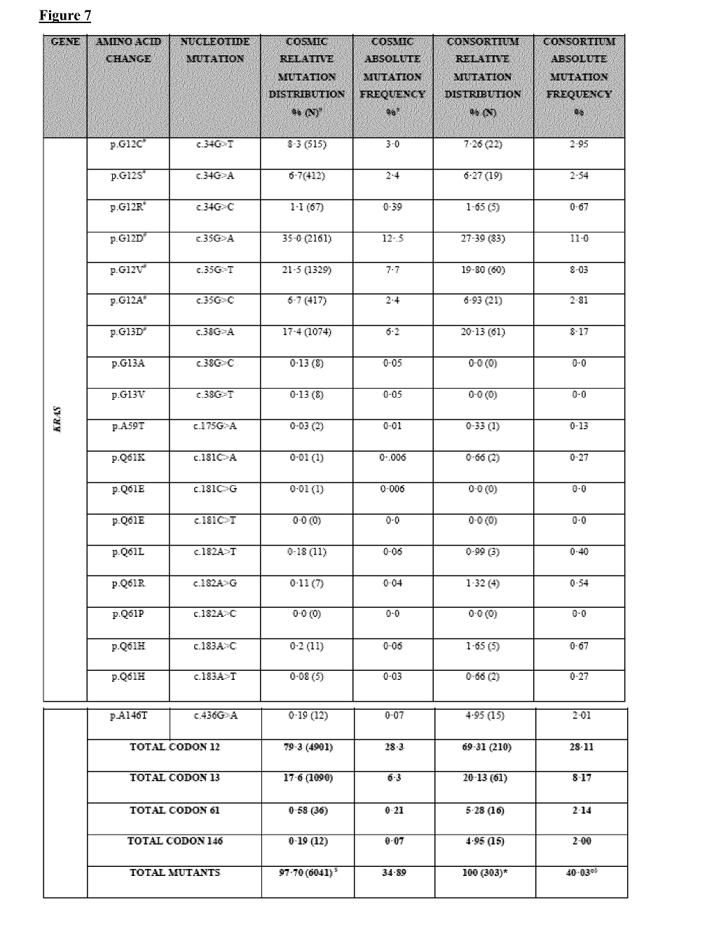

The inventors surprisingly found that a known polymorphism located in exon 20 of the EGFR gene and having the number rs1050171 according to the NCBI SNP database (http://www.ncbi.nlm.nih.gov/snp/?term=rs1050171; SEQ ID NO: 1 as depicted in FIG. 6) is such a suitable biomarker. Polymorphism rs1050171 is located in the EGFR tyrosine kinase domain at nucleotide 2607 of the corresponding EGFR mRNA, amino acid 787 (GM) and changes nucleotide 2607 from G to A, however, without an amino acid substitution. Accordingly, three genotypes may be identified, i.e. GG, AG and AA. It has now been found that patients with genotype GG at rs1050171 show a longer progression free and overall survival with respect to other patients when treated with anti-EGFR molecules. Hence, polymorphism rs1050171 is a suitable biomarker for predicting the treatment response of a patient suffering from colorectal cancer to an anti-EGFR molecule treatment.

Accordingly, the present invention relates to a method for predicting the treatment response to an anti-EGFR molecule in a patient suffering from colorectal cancer, comprising

a) providing a nucleic acid sample from the patient suffering from colorectal cancer,

b) performing a single nucleotide polymorphism (SNP) genotyping analysis at rs1050171 on said sample,

wherein genotype GG at rs1050171 is indicative for a treatment response to an anti-EGFR molecule.

The method for predicting a treatment response according to the present invention allows to determine the likelihood that a patient will exhibit a positive or negative clinical response to treatment with an anti-EGFR molecule. Such predictive methods can be used by the medicinal practitioner in order to chose the appropriate treatment regimen for any patient suffering from CRC and constitute a valuable tool for predicting whether a patient is likely to respond favorably to anti-EGFR molecule treatment.

The term “treatment response in a patient suffering from colorectal cancer” in the sense of the invention refers to a positive clinical response to the treatment in a patient having been diagnosed with CRC. This treatment response may occur during and/or after the treatment with one or more anti-EGFR molecule(s). Such a positive clinical response may range from stopping the progression of the tumor to a partial or full remission of the tumor, but also includes an increase of the time of the progression free interval, of the time of the overall survival and/or of the time of the disease free survival of CRC. Overall survival (OS) as used herein refers to the time span from starting the treatment until CRC specific death of the patient. Disease free survival refers to the time span of survival of patients having been disease free due to a treatment against colorectal cancer (e.g. by surgery, chemotherapy, anti-EGFR molecule treatment) until the next relapse. In contrast thereto, progression free interval denotes the time span after treatment during which the CRC does not worsen or progress. Treatment response, however, also includes a partial alleviation of the symptoms or a complete remission of the symptoms, indicated by a change of symptoms strength and/or frequency. Exemplary symptoms of CRC include blood in the faeces, a change of normal bowel habit to diarrhea but also to constipation, pain in the abdomen or back passage, loss of weight, fatigue and nausea. Further exemplary symptoms include symptoms indicating a recurrent CRC such as abdominal pain, dry cough, fatigue, nausea and/or unexplained weight loss.

As used herein “a patient suffering from colorectal cancer” refers to any mammalian, in particular human, patient having developed atypical and/or malignant cells in the lining and/or the epithelium of the large intestine, rectum and/or appendix. This includes CRC patients independent of the stage and form of the CRC. Patients suffering from colorectal cancer also include patients which are recurrent with colorectal cancer, i.e. patients wherein after surgical treatment the tumor could no longer be detected for a certain time span, but wherein the cancer has returned in the same or different part of the large intestine, rectum and/or appendix and/or wherein metastases have developed at different sites of the patient's body such as in the liver, lung, peritoneum, lymph nodes, brain and/or bones. In another embodiment, the patient suffering from CRC is a patient wherein the initial tumor has already been treated surgically and the CRC is non-metastatic.

CRC may be staged according to the Dukes system, the Astler-Coller system or the TNM system (tumors/nodes/metastases), whereby the latter is most commonly used. The TNM system of the American Joint Committee of Cancer (AJCC) describes the size of the primary tumor (T), the degree of lymph node involvement (N) and whether the cancer has already formed distant metastasis (M), i.e. spread to other parts of the body. Here, stages 0, IA, IB, IIA, IIB, III and IV are defined based on the determined T-, N- and M-values. A corresponding staging scheme can be derived from the Cancer Staging Manual of the AJCC (Edge et al., 2010). Another system for staging of colorectal cancer is the Dukes system established by the British pathologist Cuthbert Dukes, defining cancer stages A, B, C and D. This system was adapted by Astler and Colter, who further subdivided stages B and C (“modified Astler-Coller classification”). As used herein, CRC patient includes patients staged according to any staging system used and irrespective of the stage diagnosed.

In one embodiment of the method and the use described herein, the patient is suffering from metastatic colorectal cancer. Metastatic colorectal cancer includes any form of CRC wherein the cancer has spread from its original starting point to another part of the body such as the lymph nodes or any other organs. In particular, patients suffering from metastatic colorectal cancer include patients wherein the N value as defined in the TNM system is N1 (metastasis in) to 3 regional lymph nodes), N1a (metastasis in) regional lymph node), N1b (metastasis in 2-3 regional lymph nodes), N1c (tumor deposite(s) in the subserosa, mesentery, or non-peritonealized pericolic or perirectal tissues without regional nodal metastasis), N2 (metastasis in 4 or more regional lymph nodes), N2a (metastasis in 4 to 6 regional lymph nodes) or N2b (metastasis in 7 or more regional lymph nodes) and/or the M8 value according to the TNM system is M1 (distant metastasis), M1a (metastasis confined to one organ or site (e.g. liver, lung, ovary, non-regional node)) or M1b (metastasis in more than one organ/site or the peritoneum). This includes patients in stage IIIA, IIIB, IIIC, IVA and IVB according to the TNM system, in stage C according to the Dukes system and in stages C1, C2 and/or C3 according to the Astler-Coller system.

Different (histopathologic) forms of CRC include adenocarcinoma in situ, adenocarcinoma, medullary carcinoma, mucionous carcinoma (colloid type), signet ring cell carcinoma, squamous cell (epidermoid) carcinoma, adenosquamous carcinoma, small cell carcinoma, undifferentiated carcinoma and/or carcinoma NOS (not otherwise specified). Histological grades include GX (grade cannot be assessed), G1 (well differentiated), G2 (moderately differentiated), G3 (poorly differentiated) and G4 (undifferentiated).

In order to obtain and/or provide a nucleic acid sample from the patient suffering) from colorectal cancer, a tissue sample or blood sample may be taken from said patient. It is however understood, that any other sample derived from the patient and from which a nucleic acid sample may be obtained such as sputum may also be used. Methods for obtaining a blood sample from a patient are known in the art. For example, a blood sample may be taken from a patient by using a sterile needle. The tissue sample obtained from the patient may be a tissue sample of the colorectal tumor itself and/or of the normal colonic mucosa close to the primary tumor. Close to the primary tumor denotes a sample taken at least about 0.1 cm, about 0.2 cm, about 0.3 cm, about 0.4 cm, about 0.5 cm, about 1 cm, about 5 cm or 10 cm from the margin of the primary tumor. Methods for obtaining such tissue samples are known to the person skilled in the art and include e.g. open surgery, laparascopy and colonscopy. The step of obtaining the tissue sample is not part of the present method.

Subsequently, the DNA is extracted or purified from the sample prior to SNP genotyping analysis. Any method known in the art may be used for DNA extraction or purification. Suitable methods comprise inter alia steps such as centrifugation steps, precipitation steps, chromatography steps, dialyzing steps, heating steps, cooling steps and/or denaturation steps. For some embodiments, a certain DNA content in the sample may have to be reached. DNA content can be measured for example via UV spectrometry as described in the literature. Thus, DNA amplification may be useful prior to the SNP analysis step. Any method known in the art can be used for DNA amplification. The sample can thus be provided in a concentration and solution appropriate for the SNP analysis.

For the SNP genotyping analysis performed in step b), SNP-specific primers and/or probes, a primer extension reaction, SNP microarrays, restriction analysis and/or DNA-sequencing may be used. Reagents and methods for performing SNP genotyping analyses are known in the art.

In one embodiment of the invention, the SNP genotyping analysis performed in step b) of the method disclosed herein includes a PCR followed by restriction analysis. More specifically, after extraction of the DNA (e.g. from the normal colonic mucosa of the patient), a PCR amplification is made to cover the exon 20 of the EGFR gene (primers: forward-5′-CACACTGACGTGCCTCTC-3′ (SEQ ID NO: 2); reverse-5′-GGATCCTGGCTCCTTATCTC-3′ (SEQ ID NO: 3)). The PCR-amplicon is then submitted to restriction analysis using the ALU I enzyme according to the manufacturer's instructions. Such approach can be used because nucleotide change from G to A abolishes the restriction site of ALU I and the different genotypes may thus be identified according to changes in DNA length upon analyzing the DNA fragments after the restriction digest.

In an alternative embodiment, the SNP genotyping analysis of step b) of the method disclosed herein is performed by DNA-sequencing. DNA sequencing usually employs a primer designed as flanking the region to be analysed together with labelled nucleotides in a PCR-like setup. By analysing the labels at the corresponding positions, it is possible to determine the sequence of DNA starting from the regions to which the primer is hybridising. Furthermore, it is possible to determine the genotype of an allele by sequencing since a peak corresponding to two different bases or a peak indicating an identical base at a certain position may be detected.

DNA-microarray techniques may also be used in step b); the techniques are based on hybridisation events between the test-DNA and so-called “probes” immobilised on defined spots of a Microarray in a chamber. Today, such microarrays are routinely used to determine DNA-sequences even down to the level of a single base and thus for the detection of SNPs. This is possible by selecting the probes accordingly and using specific hybridisation conditions. The DNA may be labelled for detecting purposes. Routinely, probes covering the different sequences at the position of an SNP may be used in combination with corresponding controls; thus, also the genotype of the corresponding SNP may be analysed.

Further, real-time PCR methods may also be used in step b), wherein real-time PCR is based on the incorporation of double strand specific dyes into DNA while said DNA is amplified. Said dyes are detected only in case they are incorporated. Thus, the more DNA amplified, the higher the detection signal of the corresponding dye. By designing primers accordingly and/or by adding suited probe-nucleotides hybridising to a specific DNA-sequence only (which are able to discriminate between SNPs) and using specific hybridisation conditions, polymorphisms may be analysed.

Also, mass-spectrometry (MS) may be used in step b) of the present method. In MALDI-MS, a sample is mixed with a solution containing a matrix material and a drop of the liquid is placed on the surface of a probe. The matrix solution then e.g. co-crystallises with the biological sample and the probe is inserted into the mass spectrometer and laser energy is then directed to the probe surface where it absorbs and ionises the biological molecules without significantly fragmenting them.

In another embodiment of the method described herein the SNP genotyping analysis of step b) includes a combination of the above described methods, in particular restriction analysis with at least one further method for identifying the genotype at rs1050171 of the patient described herein, in particular DNA-sequencing.

If the result of step b) of the method described herein is, that the patient has genotype GG at rs1050171, this is indicative for a (positive) treatment response to an anti-EGFR molecule. As set out above, this means a positive clinical response to a treatment with an anti-EGFR molecule.

If, however, the patient exhibits genotype AG or AA at rs1050171, further analyses may be performed in order to determine whether the patient will show a treatment response to an anti-EGFR molecule.

In particular, it has been found that patients exhibiting genotype AG or AA at rs1050171 and showing high EGFR expression levels will also show a treatment response to treatment with an anti-EGFR molecule.

Accordingly, in one of its embodiments, the method for predicting a treatment response to an anti-EGFR molecule further comprises a step c) of determining the EGFR expression level if the SNP genotyping analysis shows genotype AG or AA at rs1050171, wherein genotypes AG or AA at rs1050171 in combination with a high EGFR expression level are indicative for a treatment response to an anti-EGFR molecule.

For determining the EGFR expression level, the levels of EGFR mRNA may be quantified. Methods for performing mRNA quantification are known to the person skilled in the art and include northern blotting, real-time quantitative PCR, serial analysis of gene expression (SAGE) and/or DNA-microarrays. Any of the aforementioned methods may be performed in order to determine the EGFR expression level in step c) of the method described herein. In one embodiment, the EGFR expression level is determined by means of real-time quantitative PCR.

Alternatively, the EGFR protein level may be analyzed in order to determine the EGFR expression level. This may be done by standard methods such as Western blotting, spectroscopic assays, colorimetric assays and immunohistochemistry by using anti-EGFR antibodies.

“High EGFR expression level” as used herein denotes any expression level of EGFR which is above the level normally found in patients. This includes EGFR mRNA expression levels and/or EGFR protein expression levels. In particular, it denotes EGFR expression levels which are 10%, 20%, preferably 30%, 40%, more preferably 50% or more above the median general value of expression in patients suffering from CRC. The median general value of EGFR expression may be determined in a group of CRC patients of at least 100 patients, 120 patients, 140 patients, 160 patients, 180 patients, 200 patients or more, whereby the patients may be chosen irrespective of their current treatment and the type of CRC. The median general value of EGFR expression may be a predetermined fixed value obtained from a group of patients as set out above. In this case, it can easily be determined whether the EGFR expression level is high, since a comparison of the obtained individual EGFR expression level to the median general value can be made and it can be determined whether the individual EGFR expression level is above the median general value.

In the following paragraphs, reference will be made to a wild-type status or a mutational status of certain genes and proteins, in particular of the genes KRAS, BRAF, PI3KCA and PTEN and the proteins encoded thereby, wherein the wild-type gene sequences are depicted in SEQ IDs NO: 12 to 15 (including exons and introns).

The term “wild-type status” as used herein refers to the wild-type amino acid sequences of the proteins KRAS (Swiss-Prot: P42336.2), BRAF (GenBank: AAA35609.2), PI3KCA (Swiss-Prot: P42336.2) and PTEN (GenBank AAD13528) (see SEQ IDs NO: 16 to 19), and to the underlying nucleotide sequence (on a DNA-level) encoding such wild-type proteins. It also refers to the status at specific amino acid positions of these proteins and the underlying specific coding nucleotides.

If e.g. the mutational state of BRAF is analyzed, this may be done by sequencing the exonic regions of the BRAF-gene in the DNA of a provided patient sample. If these regions correspond to the wild-type DNA sequence, the encoded protein will also be in the “wild-type status”. If these regions comprise one or more silent nucleotide mutations, i.e. mutations, which do not result in an amino acid exchange in the encoded protein, the encoded protein will still be in the “wild-type status”. Such silent mutations may not be classified as “mutational status” in the present application. If however, these regions comprise one or more nucleotide exchanges, which result in at least one amino acid exchange of the encoded protein, such a status is referred to as “mutational status” of a gene. Accordingly, a mutation resulting in an amino acid exchange at a specific position of the KRAS protein is also defined as “mutational status” in the present invention.

Generally, a mutational state of the genes discussed below (in the meaning of a non-wild-type encoded protein as defined above comprising at least one amino acid exchange) may be indicative for a response discussed in the following. Preferably, a mutational state of the genes is linked to specific amino acid substitutions, as will be discussed below. A heterozygous mutational state of the genes discussed below is already sufficient for the method of the present invention. Particularly in the case of PTEN, a mutational status may also be understood as complete loss of expression of the corresponding gene such that no protein is detectable at all. This may be due to deletion of the gene or inactivating epigenomic marks, such as methyl-marks, particularly in the promoter region.

In order to further define the likelihood for a treatment response of a patient, the method described herein may further include a step d) of determining the KRAS mutational status. Thus, in another embodiment of the invention, the KRAS mutational status is determined for the patient exhibiting genotype GG at rs1050171. In an alternative embodiment of the invention, the KRAS mutational status is determined for a patient exhibiting genotype AG or AA at rs1050171 and showing a high expression level of EGFR.

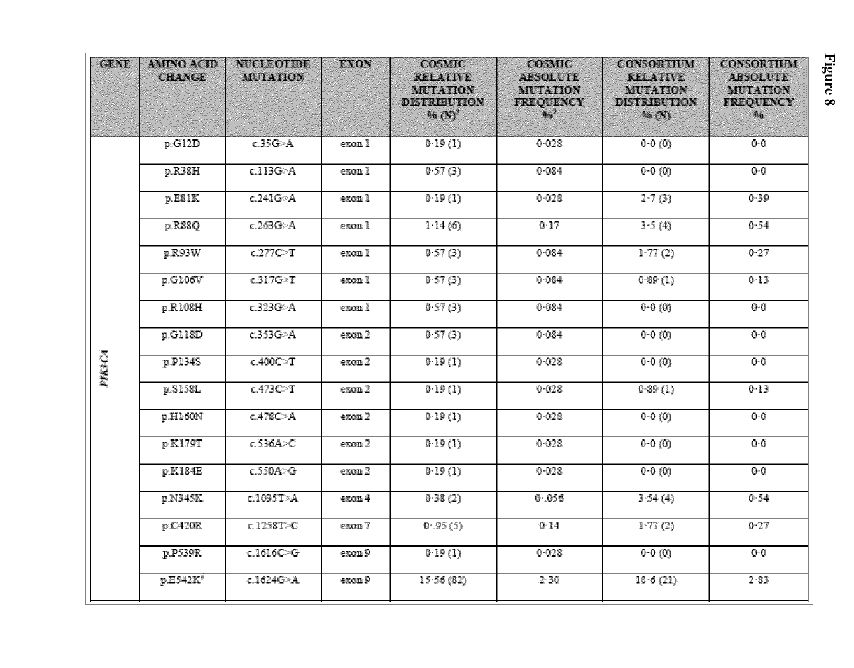

The KRAS gene is a member of the RAS family and functions in coupling signal transduction from surface receptors to intracellular targets. While RAS signaling is normally tightly regulated, mutant KRAS proteins are characterized by constitutive activation of RAS signaling, thus leading to stimulation of the MAPK pathway which is independent of EGFR. As a result, blockade of EGFR does not alter downstream signaling of the MAPK pathway in cells with mutant KRAS and has no affect on cell growth, proliferation, or survival. Thirty to forty percent of colorectal cancers contain a mutated KRAS gene. The most common KRAS mutations result in amino acid exchanges at positions 12 or 13 (a G is found in the wild-type status at these two positions, see SEQ ID NO: 13), although some mutations also result in amino acid exchanges at positions 61 and 146 (Q and A, respectively, are found in the wild-type status at these two positions, see SEQ ID NO: 13). Multiple studies have demonstrated that the presence of such KRAS mutations results in a lack of response to the anti-EGFR monoclonal antibodies, and that all favourable responses occur in a subset of the patients whose tumors exhibit a wild-type KRAS status. Exemplary KRAS mutations resulting in a mutational status as defined for the present invention are depicted in FIG. 7 (see nucleotide mutations at the positions 34, 35, 38, 175, 181, 182, 183 and 436; the resulting amino acid changes are also depicted in FIG. 7), while the wild-type KRAS gene is accessible under NCBI accession number NG_{—}007524.1 (SEQ ID NO: 12).

Hence, in one embodiment of the method described herein, the KRAS mutational status resulting in amino acid substitutions at positions 12, 13, 59, 61 and/or 146 is determined, whereby a wild-type status at any of these positions can be indicative for a treatment response to an anti-EGFR molecule. In an even preferred embodiment, the KRAS mutational status resulting in the amino acid changes G12C, G12S, G12R, G12D, G12V, G12A, G13D, G13A, G13V, A59T, Q61K, Q61E, Q61L, Q61R, Q61P, Q61H and/or A146T is determined, whereby a wild-type status at any of these positions may be indicative for a treatment response to an anti-EGFR molecule.

A few studies have also raised the possibility that the KRAS G13D change and a change at amino acid position 146 do not confer the same degree of resistance to EGFR inhibitors, although additional studies are required to corroborate these findings. Thus, in another preferred embodiment, the KRAS mutational status resulting in the amino acid changes G12C, G12S, G12R, G12D, G12V, G12A, G13A, G13V, A59T, Q61K, Q61E, Q61L, Q61R, Q61P and/or Q61H is determined, whereby a wild-type status at any of these positions may be indicative for a treatment response to an anti-EGFT molecule. In yet another preferred embodiment, the KRAS mutational status resulting in the amino acid changes G12C, G12S, G12R, G12D, G12V, G12A, G13A, G13V and/or A146T is determined, whereby a wild-type status at any of these positions is indicative for a treatment response to an anti-EGFR molecule.

In a particularly preferred embodiment, the KRAS mutational status resulting in amino acid substitutions at positions 12 and/or 13 and/or 61 and/or 146 is determined, whereby a wild-type status at these positions is indicative for a treatment response to an anti-EGFR molecule. It is particularly preferred to determine the KRAS mutational status resulting in amino acid substitutions at positions 12 and/or 13, whereby a wild-type status at these positions is indicative for a treatment response to an anti-EGFR molecule.

To date, the validation of KRAS mutation status as a predictive molecular marker of non-response to EGFR-targeted drugs has been one of the most important developments in molecular markers for metastatic CRC. Consequently, the American Society of Clinical Oncology (ASCO) guidelines recommend that KRAS gene mutation analysis be performed as part of the pre-treatment workup in all patients with metastatic CRC before initiating anti-EGFR therapy.

Since favourable responses to anti-EGFR molecule treatment are found in patients having wild-type KRAS, in particular wild type KRAS at amino acid positions 12 and/or 13, genotypes AG or AA at rs1050171 in combination with a high EGFR expression level and in combination wild-type KRAS may be particularly indicative for a treatment response to an anti-EGFR molecule.

In another embodiment of the method according to the invention, genotype GG at rs1050171 in combination with wild-type KRAS, in particular wild-type KRAS at amino acid positions 12 and/or 13 is indicative for a treatment response to an anti-EGFR molecule.

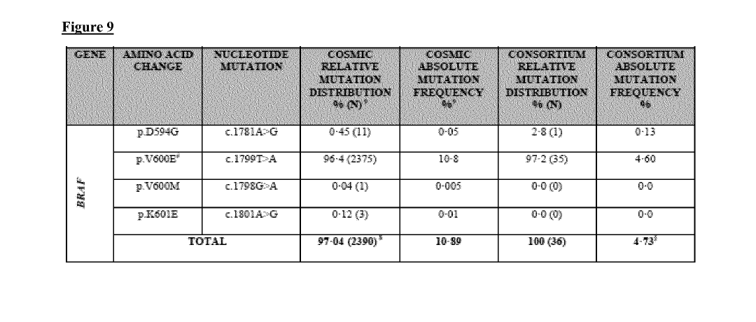

BRAF is the immediate downstream effector of KRAS in the MAPK pathway and mutations in this gene (mainly the somatic V600E mutation) occur in approximately 15% of CRCs (Saridaki et al., 2010). BRAF mutations are mutually exclusive with KRAS mutations, and they may activate the signaling pathway in a similar manner to KRAS mutations. Few studies have shown that KRAS wild-type, BRAF mutant CRCs may be resistant to EGFR inhibitors (Di Nicolantonio et al., 2008), although not all found this as being a robust relationship. BRAF mutations, indeed, appear to be associated with worse prognosis independent of treatment, showing therefore a prognostic relevance (Tol et al., 2009). Exemplary BRAF mutations resulting in a mutational status as defined for the present invention are depicted in FIG. 9 (see nucleotide mutations at the positions 1781, 1799, 1798 and 1801; the resulting amino acid changes are also depicted in FIG. 9), while the wild-type BRAF gene sequence is accessible under NCBI accession number M95712.2 (SEQ ID NO: 13).

Thus, in another embodiment of the invention, the method includes alternatively or additionally to step d) a further step of determining the BRAF mutational status, particularly the wild-type status at amino acid positions 549, 600 and/or 601, whereby a wild-type at BRAF at any of these positions may indicate a positive treatment response to an anti-EGFR molecule (which may be denoted step e)).

In one embodiment of the invention, the BRAF mutational status is determined for the patient exhibiting genotype GG at rs1050171. In an alternative embodiment of the invention, the BRAF mutational status is determined for a patient exhibiting genotype AG or AA at rs1050171 and showing a high EGFR expression.

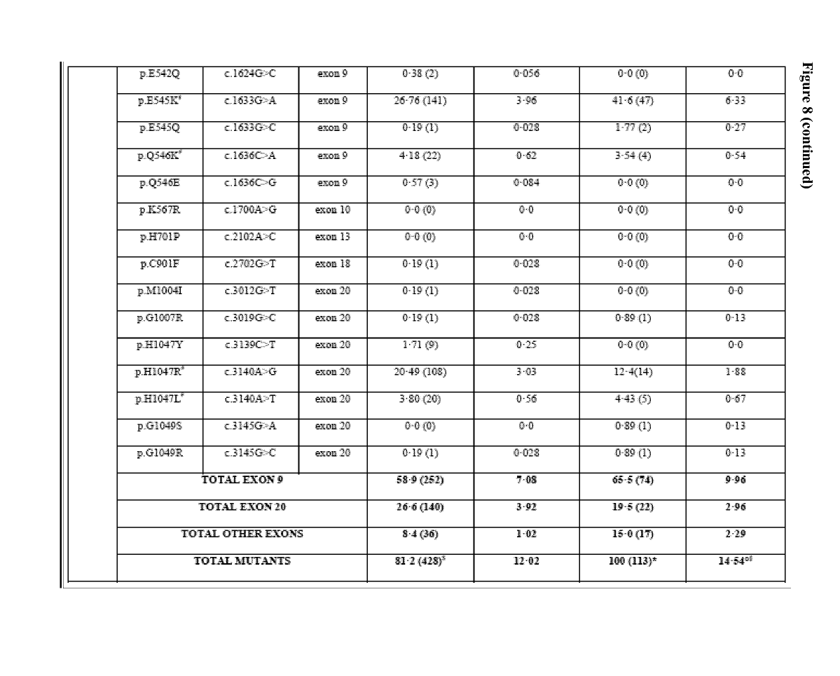

The PI3KCA gene is mutated in about 20% of CRCs. It encodes for the p110α subunit of PI3K, a lipid kinase which regulates, alongside with KRAS, signalling pathways downstream of the EGFR. “Hotspot” mutations in PI3KCA gene are localized at exon 9 and exon 20. PIK3CA mutations were significantly associated with clinical resistance to Panitumumab or Cetuximab and patients with PIK3CA mutations displayed a worse clinical outcome also in terms of progression-free survival (Sartore-Bianchi et al., 2009b). Exemplary PIK3CA mutations resulting in a mutational status as defined for the present invention are depicted in FIG. 8 (see nucleotide mutations at the positions 35, 113, 241, 263, 277, 317, 323, 353, 400, 473, 478, 536, 550, 1035, 1258, 1616, 1624, 1633, 1636, 1700, 2102, 2702, 3012, 3019, 3139, 3140 and 3145; the resulting amino acid changes are also depicted in FIG. 8), while the wild-type PIK3CA gene sequence is accessible under NCBI accession number NM_{—}006218.2 (SEQ ID NO: 14).

Thus, in another embodiment of the invention, the method includes alternatively or additionally to step d) a further step of determining the PI3KCA mutational status, whereby a wild-type status of PI3KCA, in particular a wild-type status of PI3KCA at exon 9 and/or exon 20 indicates a positive treatment response to an anti-EGFR molecule (which may be denoted step f)).

In one embodiment of the invention, the PI3KCA mutational status is determined for the patient exhibiting genotype GG at rs 1050171. In an alternative embodiment of the invention, the PI3KCA mutational status is determined for a patient exhibiting genotype AG or AA at rs1050171 and showing a high EGFR expression.

PTEN is a phosphatase that inhibits signalling initiated by PI3K. Therefore, loss of PTEN could result in activation of PI3K signalling and resistance to EGFR inhibitors. PTEN expression is decreased in about 20% of CRCs and it has been associated with lack of response to Cetuximab (Bardelli and Siena, 2010; Sartore-Bianchi et al., 2009a). The wild-type PTEN gene sequence is accessible under NCBI accession number NM_{—}000314.4 (SEQ ID NO: 15).

Thus, in another embodiment of the invention, the method alternatively or in addition to step(s) e) and/or f) includes a step g) of determining the PTEN mutational status, whereby a mutation at PTEN, in particular a mutation leading to a loss of function and/or loss of expression of PTEN, indicates a negative treatment response to an anti-EGFR molecule.

As outlined above, a loss of expression may particularly for PTEN also be understood as mutational status, wherein the expression and a loss of expression, respectively, of the PTEN-protein (SEQ ID No: 19; see above) may be determined with methods outlined above. Thus, in another embodiment of the invention, the method alternatively or in addition to step(s) e) and/or f) includes a step g) of determining the PTEN protein expression wherein PTEN-expression and thus the presence of PTEN protein (usually detected by IHC) indicates a positive treatment response to an anti-EGFR molecule.

In one embodiment of the invention, the PTEN status is determined for the patient exhibiting genotype GG at rs1050171. In an alternative embodiment of the invention, the PTEN status is determined for a patient exhibiting genotype AG or AA at rs1050171 and showing a high EGFR expression. In these two embodiments regarding the PTEN status, the term “status” preferably refers to the status of expression of the PTEN-protein.

Methods for determining the mutational status are known to the person skilled in the art and include amongst others DNA sequencing, real-time PCR with specific primers and probes, RT-PCR, fluorescence in situ hybridisation, immunohistochemistry, semi-nested PCR and/or nested PCR. In one embodiment of the invention, the mutational status, in particular the KRAS mutational status is determined via semi-nested PCR and/or DNA sequencing.

As has been described herein above, it has been found that the specific group of CRC patients defined herein shows a treatment response to anti-EGFR molecules.

Thus, a further aspect of the present invention relates to an anti-EGFR molecule for use in the treatment of a patient suffering from colorectal cancer, wherein the patient exhibits

a) genotype GG at rs1050171 or

b) genotype AG or AA at rs1050171 and a high expression level of EGFR.

A patient exhibiting a high mRNA expression level of EGFR includes any patient showing mRNA and/or protein expression levels which are 10%, 20%, preferably 30%, 40%, more preferably 50% or more above the median general value of expression in patients suffering from CRC. In one embodiment, the median general value of EGFR expression may be determined in a group of CRC patients of at least 100 patients, 120 patients, 140 patients, 160 patients, 180 patients, 200 patients or more, whereby the patients may be chosen irrespective of their current treatment and the type of CRC.

Methods for determining the genotype at rs1050171 and the EGFR expression levels (particularly mRNA and protein levels) have been described above.

In one embodiment according to the invention the patient to be treated with the anti-EGFR molecule as defined under item a) or b) further exhibits a wild-type KRAS status, in particular a wild-type KRAS status at amino acid positions 12 and/or 13. In a further embodiment of the invention, the patient to be treated with an anti-EGFR molecule as defined under item a) or b) exhibits a wild-type KRAS status at amino acid positions 13 and/or 61 and/or 146. In another embodiment according to the present invention, the patient as defined under item a) or b) exhibits a wild-type KRAS status at amino acid position 12, 13, and/or 61 and 146.

In another embodiment, the patient to be treated with the anti-EGFR molecule as defined under item a) or b) exhibits a wild-type BRAF status.

In a further embodiment, the patient to be treated with the anti-EGFR molecule as defined under item a) or b) exhibits a wild-type PIKCA status, in particular a wild-type PI3KCA status at exon 9 and/or exon 20.

In a further embodiment, the patient to be treated with the anti-EGFR molecule as defined under item a) or b) exhibits a wild-type PTEN status and preferably a regular PTEN protein expression status.

It is understood herein, that the patient to be treated with the anti-EGFR molecule as defined under item a) or b) may exhibit a wild-type status of KRAS, a wild-type status of BRAF, a wild-type status of PI3KCA and/or a wild-type status (and/or expression) of PTEN protein.

Further, it is to be understood that the patient to be treated with an anti-EGFR molecule may solely be treated with said anti-EGFR molecule or additionally with an adjuvant therapy, such as e.g. a chemotherapy based on 5-FU, Oxaliplatin and/or Irinotecan.

“Anti-EGFR molecules” as used herein refers to any compound capable of interfering with the expression and/or function of EGFR. Compounds interfering with the function of EGFR are compounds which bind directly or indirectly to the EGFR so as to modulate the receptor mediated activity, while compounds interfering with the expression of EGFR relates to compounds interfering at any stage of EGFR gene expression so as to reduce the number of EGFR obtained. Anti-EGFR molecules according to the present invention include anti-EGFR antibodies, small molecules directed to EGFR and inhibitory polynucleotides capable of interfering with the expression and/or function of EGFR. Anti-EGFR molecules generally include any anti-EGFR molecule which can be used for CRC therapy such as anti-EGFR molecules, in particular anti-EGFR antibodies, small molecules and inhibitory polynucleotides which were tested in clinical trials as well as anti-EGFR molecules currently studied in clinical trials and/or to be developed. Exemplary anti-EGFR molecules which have already been tested in clinical trials and approved to the market include the anti-EGFR antibodies Cetuximab and Panitumumab as well as the small molecules Erlotinib and Gefitinib.

In one embodiment of the method as well as of the use described herein, the anti-EGFR molecule is an anti-EGFR antibody. Anti-EGFR antibodies denote any antibody or fragment thereof that binds specifically to EGFR. It is understood that “binds specifically” or “specifically binding” can relate to an antibody having a binding affinity to the EGFR of ≦10⁻⁹ mol/l, particularly of ≦10⁻¹⁰ mol/l. Methods for determining the binding affinity of antibodies to antigens are known in the art and include e.g. the use of surface plasmon resonance.

In the context of the present invention the term “antibody” relates to full length antibodies, human antibodies, humanized antibodies, fully human antibodies, genetically engineered antibodies and multispecific antibodies, as well as to fragments of such antibodies retaining the characteristic properties of the full length antibody. In one embodiment of the method as well as of the use described herein, the antibody is a humanized antibody. A “humanized antibody” is an antibody which has been modified in order to provide an increased similarity to antibodies produced in humans, e.g. by grafting a murine CDR into the framework region of a human antibody. In another embodiment of the method as well as of the use described herein, the antibody is a fully human antibody.

Anti-EGFR antibodies may be monoclonal or polyclonal antibodies. Monoclonal antibodies are monospecific antibodies (i.e. binding to the same epitope) derived from a single cell line. Hence, monoclonal antibodies are, except for variants arising during their production, substantially identical antibodies. In contrast thereto, polyclonal antibodies relates to a variety of antibodies directed to different epitopes of an antigen. Methods for production of monoclonal and polyclonal antibodies are known in the art and include e.g. the hybridoma technology and recombinant DNA methods. In one embodiment of the method as well as of the use described herein, the anti-EGFR antibody is a monoclonal antibody.

In a further embodiment of the method as well as of the use described herein, the anti-EGFR antibody is selected from the group consisting of Cetuximab and Panitumumab. The monoclonal antibodies Cetuximab (Erbitux®) and Panitumumab (Vectibix®) compete with natural ligands and block EGFR activation, thus inhibiting growth of CRC cells. Panitumumab is a fully human monoclonal antibody specific to EGFR, while Cetuximab is a chimeric (mouse/human) monoclonal antibody.

Further anti-EGFR molecules according to the present invention include small molecules directed to EGFR. Small molecules directed to EGFR include any organic compound having a low molecular weight, in particular a molecular weight not exceeding 800 Da, not being a polymer and capable to bind to EGFR, thus interfering with its function. In one embodiment of the method as well as of the use described herein, the small molecule directed to EGFR is selected from the group consisting of Erlotinib and Gefitinib.

In a further embodiment of the method as well as of the use described herein, the anti-EGFR molecule is an inhibitory polynucleotide molecule capable of interfering with the expression and/or function of EGFR. Such inhibitory polynucleotides include antisense oligonucleotide specific for EGFR, small interfering RNA (siRNA) specific for EGFR, or a microRNA specific for EGFR.

The term “antisense oligonucleotide specific for EGFR” refers to nucleic acids corresponding to complementary strand of the EGFR mRNA. Preferably, the antisense oligonucleotide comprises a sequence complementary to at least a portion of the EGFR gene expression product. Generally, antisense technology can be used to control, i.e. reduce or abolish gene expression through antisense DNA or RNA, or through triple-helix formation. In one embodiment, an antisense molecule may be generated internally by the organism, for example intracellularly by transcription from an exogenous sequence. A vector or a portion thereof may be transcribed, producing an antisense nucleic acid of the invention. Such a vector can remain episomal or become chromosomally integrated, as long as it can be transcribed to produce the desired antisense molecule. Corresponding vectors can be constructed by recombinant DNA technology methods known to the person skilled in the art. Vectors can be plasmid, viral, or others known in the art, used for replication and expression in vertebrate cells, e.g. vectors as defined herein above.

The term “siRNA specific for EGFR” as mentioned herein above refers to a particular type of small molecules, namely small inhibitory RNA duplexes that induce the RNA interference (RNAi) pathway to negatively regulate gene expression of EGFR. Methods for designing suitable siRNAs directed to a given target nucleic acid are known to person skilled in the art.

The term “miRNA specific for EGFR” as used herein refers to a short single-stranded RNA molecule of typically 18-27 nucleotides in length, which regulate gene expression of EGFR. miRNAs are encoded by genes from whose DNA they are transcribed but are not translated into a protein. Mature miRNA molecules are typically at least partially complementary to mRNA molecules corresponding to the expression product of the present invention, and fully or partially down-regulate gene expression. Preferably, miRNAs according to the present invention may be 100% complementary to their target sequences. Alternatively, they may have 1, 2 or 3 mismatches, e.g. at the terminal residues or in the central portion of the molecule.

Another aspect of the present invention relates to a kit or diagnostic composition for the analysis of a single nucleotide polymorphism indicative for the treatment response to an anti-EGFR molecule, comprising at least one primer and/or probe for determining the genotype at rs1050171.

The term “primer” as used herein denotes an oligonucleotide that acts as an initiation point of nucleotide synthesis under conditions in which synthesis of a primer extension product complementary to a nucleic acid strand is induced.

The term “probe” as used herein denotes an oligonucleotide that selectively hybridizes to a target nucleic acid under suitable conditions.

The primers and probes may be generated such that they are able to discriminate between wild-type allele or mutated allele of the position of the SNP to be analyzed, i.e. of rs1050171. Methods for the design of sequence specific primers and probes are known in the art. Exemplary primers which may be used are those shown in SEQ ID NOs: 4 to 7.

As used herein, a kit relates to a product containing reagents necessary for determining the treatment response to anti-EGFR molecules in CRC patients (e.g. a diagnostic composition) which are packed so as to allow their transport and storage. The kit may further contain a package leaflet describing how the kit and its components should be used.

Diagnostic composition as used herein may relate to any composition allowing to determine the genotype at rs1050171 and comprising at least one primer and/or probe for determining said genotype.

Thus, such a kit or diagnostic composition for the analysis of a SNP indicative for the treatment response to an anti-EGFR molecule may further comprise one or more enzymes for primer elongation, nucleotides and/or labeling agents.

Further aspects and embodiments of the invention are set out in the below items:
1. Method for predicting the treatment response to an anti-epidermal growth factor receptor (EGFR) molecule in a patient suffering from colorectal cancer, comprising
   a) providing a nucleic acid sample from the patient suffering from colorectal cancer,
   b) performing a single nucleotide polymorphism (SNP) genotyping analysis at rs1050171 on said sample, wherein genotype GG at rs1050171 is indicative for a treatment response to an anti-EGFR molecule.
2. The method according to item 1, further comprising step c) of determining the EGFR expression level if the SNP genotyping analysis shows genotype AG or AA at rs1050171, wherein genotypes AG or AA at rs1050171 in combination with a high EGFR expression level are indicative for a treatment response to an anti-EGFR molecule.
3. The method according to item 2, further comprising step d) of determining the KRAS mutational status, wherein genotypes AG or AA at rs1050171 in combination with a high EGFR expression level and wild-type KRAS status are indicative for a treatment response to an anti-EGFR molecule.
4. The method according to any of the preceding items, wherein the anti-EGFR molecule for which a treatment response is to be predicted is selected from the group consisting of anti-EGFR antibodies, small molecules directed to EGFR and inhibitory polynucleotides capable of interfering with the expression and/or function of EGFR.
5. The method according to item 4, wherein the anti-EGFR molecule is an anti-EGFR antibody.
6. The method according to item 5, wherein the anti-EGFR antibody is selected from the group consisting of Cetuximab and Panitumumab.
7. The method according to item 4, wherein the anti-EGFR molecule is a small molecule directed to EGFR.
8. The method according to item 7, wherein the small molecule directed to EGFR is selected from the group consisting of Erlotinib and Gefitinib.
9. The method according to any of the preceding items, wherein the colorectal cancer is metastatic colorectal cancer.
10. Anti-EGFR molecule for use in the treatment of a patient suffering from colorectal cancer, wherein the patient exhibits
   a) genotype GG at rs1050171 or
   b) genotype AG or AA at rs1050171 and a high expression level of EGFR.
11. The anti-EGFR molecule for use according to item 10, wherein the patient as defined under item b) exhibits a wild-type KRAS status.
12. The anti-EGFR molecule for use according to item 10 or 11, wherein the anti-EGFR molecule is selected from the group consisting of anti-EGFR antibodies, small molecules directed to EGFR and inhibitory polynucleotides capable of interfering with the expression and/or function of EGFR.
13. The anti-EGFR molecule for use according to item 12, wherein the anti-EGFR molecule is an anti-EGFR antibody.
14. The anti-EGFR molecule for use according to item 13, wherein the anti-EGFR antibody is selected from the group consisting of Cetuximab and Panitumumab.
15. The anti-EGFR molecule for use according to item 12, wherein the anti-EGFR molecule is a small molecule directed to EGFR.
16. The anti-EGFR molecule for use according to item 15, wherein the small molecule directed to EGFR is selected from the group consisting of Erlotinib and Gefitinib.
17. The anti-EGFR molecule according to any of items 10-16, wherein the colorectal cancer is metastatic colorectal cancer.
18. Kit or diagnostic composition for the analysis of rs1050171 as single nucleotide polymorphism indicative for the treatment response to an anti-EGFR molecule, comprising at least one primer and/or probe for determining the genotype at rs1050171.

The invention is further described in the following examples which are solely for the purpose of illustrating specific embodiments of the invention, and are also not to be construed as limiting the scope of the invention in any way.

### EXAMPLES

Material and Methods

Selection of Case Studies

We collected a case study of 98 patients with histologically confirmed metastatic colorectal cancer to study a new molecular marker of therapy response to the biological agents Cetuximab and/or Panitumumab. Tissue samples of the primary colorectal tumors were taken for the analysis. 93 patients were treated with Cetuximab-based regimes and five patients received Panitumumab, with different schedules, from October 2005 to December 2010. The patients were followed up from the date of the beginning of the therapy with the antibodies to the date of the first evidence of tumor progression or death or until 30 Apr. 2011. Clinical end points of the study were progression free survival (PFS) that was defined as the time from the start of the therapy until disease progression and overall survival (OS) that was defined as the time from start of the therapy until colon cancer specific death.

To evaluate if the results obtained in this case study were related to the treatment with the antibodies or not, we collected another case study composed of 65 patients with recurrent colorectal cancer, wherein the patients had not received a therapy with the antibodies (also referred to as “biological agents” in the following). They were selected because they had a diagnosis of recurrent colorectal cancer from February 2000 to April 2005 but did not receive the biological therapy. This latter group was followed up from the date of beginning of a standard chemotherapy (without monoclonal antibodies), and mainly based on FOLFIRI and FOLFOX 4 regimens, to the date of cancer specific death. Clinical end point was OS as above described.

DNA Extraction from FFPE

In both case studies DNA was extracted from formalin fixed and paraffin embedded (FFPE) tissues of each patient's tumor and distal normal mucosa. The areas of interest were identified on a reference H&E-stained section by a pathologist and then mechanically microdissected on the paraffin block ensuring the presence of adequate neoplastic or normal tissue. For each sample, a mean of 10-15 sections (6-8 μm thick) were cut. The dissected specimens were deparaffinized with xylene and rehydrated with ethanol. DNA was extracted according to previously published protocols (Stanta, 2011). In detail, after deparaffinization and rehydration, samples were digested in 150-300 μL of Proteinase K 1 mg/ml diluted in the appropriate digestion buffers (usually 50 mM Tris HCl pH 7.5, 1 mM EDTA, 100 mM NaCl, 0.5% Tween 20). Digestion was performed for 48-72 h at 55° C. DNA was then extracted by pH 8 buffered-phenol/chloroform and precipitated by the addition of absolute ethanol. DNA was resuspended in the appropriate amount of 1× TE buffer. Purified DNA was stored at −20° C. in aliquots.

Mutation Analyses of KRAS

We searched for KRAS point mutations in exon 2, because this exon includes mutations at codons 12 and 13. The large majority of the mutations of this gene occur in these two sites (Di Nicolantonio et al., 2008).

We performed a semi-nested PCR and mutations were detected by direct sequencing of the inner PCR product, using the forward primer of the second PCR round as the sequencing oligo (see primers below). Dideoxy sequencing reactions and sequencing runs were performed at the genomics sequencing core facility under standard conditions.

In detail, 100 ng of genomic DNA were amplified in 50 μL of final reaction volume containing 1× PCR Buffer (10 mM Tris pH 8.3; 50 mM KCl, 1.5 mM MgCl2), 0.2 mM dNTPs, 15 pmol of each appropriate primer and 1.25 units of Taq DNA Polymerase (GE Healthcare). PCR amplifications were performed as follows: initial denaturation step of 95° C. for 3′; 45 cycles of 95° C. for 30 s; specific annealing temperature for 30 s; 72° C. for 30 s; and a final elongation step of 72° C. for 5′. One μL of the first PCR reaction product was used in the second PCR round. Thermal profile of this latter was the same as the first, despite the final number of cycles (35 cycles).

The list of primers used for mutational analyses are given in Table 1.

TABLE 1

| | | | Amplicon |
|---|---|---|---|
| | | | length |
| GENE | Primers sequences | Ta | (bp) |
| KRAS | Forward: | 55° C. | 190 |
| (first | TTAACCTTATGTGTGACATGTTCT | | |
| round) | (SEQ ID NO: 4) | | |
| | Reverse: | | |
| | CAAGATTTACCTCTATTGTTGGAT | | |
| | (SEQ ID NO: 5) | | |
| KRAS | Forward: | 55° C. | 171 |
| (second | TTAACCTTATGTGTGACATGTTCT | | |
| round) | (SEQ ID NO: 6) | | |
| | Reverse: | | |
| | TGGATCATATTCGTCCACAA | | |
| | (SEQ ID NO: 7) | | |

Analysis of New Marker for Anti-EGFR Therapy

We studied an EGFR DNA sequence polymorphism that may be linked to a better response of a patient with recurrent CRC receiving therapy with Cetuximab and/or Panitumumab. This polymorphism is located in the EGFR tyrosine kinase domain at nucleotide 2607 of the corresponding EGFR mRNA, codon 787 (Gln), and it changes nucleotide 2607 from G to A, but without an amino acid substitution (silent mutation). Three genotypes may be identified: GG, AG and AA. Normal colon mucosa specimens present in the surgical tissues close to the primary tumor were used in the present study as tissue samples for EGFR-analysis.

The method used for the detection of such genotypes was PCR followed by restriction analysis. After the extraction of the DNA from the normal colonic mucosa of the patients, a PCR amplification was made to cover the exon 20 of the EGFR gene (primers: forward-5′-CACACTGACGTGCCTCTC-3′ (SEQ ID NO: 2); reverse-5′-GGATCCTGGCTCCTTATCTC-3′ (SEQ ID NO: 3)). The PCR-amplicon was then submitted to restriction analysis using the ALU I enzyme according to the manufacturer's instructions. Such approach can be used because the nucleotide change from G to A abolishes the restriction site of ALU I and the different genotypes may thus be identified according to changes in DNA length after enzyme restriction. Alternatively, the genotype was detected by sequencing the same amplicon.

RNA Extraction from FFPE

Total RNA was extracted from FFPE specimens of primary colorectal cancers from both case studies using a proteinase K-based protocol (Stanta et at., 1998). For every paraffin embedded block, 10 to15 microtome sections (6-8 μm thick) were deparaffinized with xylene and rehydrated with ethanol. When peritumoral component was present, the paraffin block was manually microdissected and only the tumor was collected. Samples were then digested in 150-400 μL of RNA digestion buffer containing 6 mg/ml proteinase K, 1.12 M Guanidine thiocyanate, 20 mM Tris HCl pH 7.5, 0.5% N-Lauroyl Sarcosine, 40 mM P-mercaptoethanol at 55° C. overnight. Total RNA was purified by acid phenol/chloroform extraction followed by ethanol precipitation. Total RNA was resuspended in the appropriate volume of DEPC treated water (between 15 and 30 μL, depending on the amount of starting tissue). Purified RNA was stored at −80° C. in aliquots.

DNAse Treatment and Reverse Transcription

For each sample, 8 μg of total RNA were digested with DNase for 15′ at 25° C. in 20 μL final volume containing 5U of DNAse I (GE Healthcare) and 1× DNase buffer (40 mM Tris-HCl, pH 7.5, 6 mM MgCl2). The enzyme was blocked with 2 μL of 25 mM EDTA and heat inactivated at 65° C. for 10′.

DNase treated RNA was then reverse-transcribed into cDNA. The RT reaction was performed using Moloney Murine leukemia virus (MMLV) reverse transcriptase and random hexamers (Nardon et al., 2009). Briefly, 2 μg of total digested RNA was added to 3.35 nmoles of random hexamers in a final volume of 9 μL. The mixture was incubated at 65° C. for 10′ and then immediately chilled on ice. At this point 11 μL of the RT mixture were added, yielding a final concentration of 1× First Strand Buffer (50 mM Tris-HCl pH 8.3; 75 mM KCl; 3 mM MgCl2—Invitrogen), 10 mM DTT (Invitrogen), 4 units of Rnase Inhibitor (Promega) 4.5 mM MgCl2, 1 mM dNTPs (Amersham) and 250 units of MMLV enzyme (Invitrogen). The mixture was left at room temperature (25° C.) for 10′, then reverse transcription was carried out at 37° C. for 50′. The enzyme was then blocked by heating at 70° C. for 10′. cDNA was stored at −20° C. in aliquots.

EGFR Gene Expression Analyses

Quantitative real time PCR was used in both case studies to quantify the mRNA transcripts of the genes of interest. To correct for quantification errors depending on differences in sample-to-sample RNA quality, GAPDH expression was assessed as normalization factor. GAPDH was chosen as reference gene in colorectal cancer according to our previous findings (Donada et al., 2010). For every target gene intron-spanning primers were designed, in accordance with specific requirements of length (between)5 and 25 bases), G/C content (around 50%), similar melting temperatures, low self-primer and hetero-primer formation and amplicon length between 60 and 100 base pairs. Syber Green chemistry was used as the detection system of amplification.

TABLE 2

| | | | | Amplicon | PCR |
|---|---|---|---|---|---|
| GENE | Primers sequences | Ta | Tf | length (bp) | efficiency |
| GAPDH | Forward: | 61° C. | 80° C. | 75 | 97% |
| | CCCTCAACGACCACTTTGTCA | | | | |
| | (SEQ ID NO: 8) | | | | |
| | Reverse: | | | | |
| | GGTCCACCACCCTGTTGCT | | | | |
| | (SEQ ID NO: 9) | | | | |
| EGFR | Forward: | 54.5 | 78.8 | 69 | |
| | GGCTCTGGAGGAAAAGAAAG | | | | |
| | (SEQ ID NO: 10) | | | | |
| | Reverse: | | | | |
| | TCAAAAGTGCCCAACTGCTG | | | | |
| | (SEQ ID NO: 11) | | | | |

Amplifications were performed using a Mastercycler® ep realplex (Eppendorf, Hamburg, Germany). All samples' amplifications were run in duplicate using the RealMasterMix SYBR ROX 2.5× (5Prime GmbH, Hamburg, Germany) according to the manufacturer's instructions. For each PCR reaction, 40 ng of cDNA were used in a final volume of 20 μl. Cycling conditions were as follows: 1′ and 30 s at 95° C. for polymerase activation and 40 cycles consisting of denaturation for 30 s at 95° C., primer annealing for 30 s at the specific temperature, extension for 30 s at 72° C. and fluorescence detection for 20 s at the specific temperature. The detection temperature was set very close to that of amplicon's melting, in order to avoid the detection of unspecific products. Uniqueness of amplification products was checked by melting curve analysis and by 10% polyacrlyamide gel electrophoresis.

In each sample, gene expression levels were normalized against the chosen housekeeping gene (GAPDH) and expressed as a fraction of that gene expression to a pool of 10 normal colon tissues, according to a ΔΔCt model previously reported (Pfaffl, 2001).

Efficiencies of real time amplification for the analyzed gene were checked in preliminary experiments plotting Ct values of PCR amplified serial dilutions of cDNAs, against the log 10 of the theorical initial RNA quantity. Efficiency was definded as 10̂(−1/slope), where the slope is obtained from the linear regression line fitted thought the points determined.

Statistics

Associations between clinical-pathological data and categories of markers were tested for significance using the chi-square test (or Fisher's exact test if any of the cells counted less than 5) for categorical variables. For continuous variables the parametric Student's t-test or the nonparametric Mann-Whitney test were used. The distribution of data within a continuous variable was tested by kurtosis test, in order to establish the type of statistical tests (parametric or non-parametric) to use. When evaluating more than two groups, the one-way ANOVA combined with Scheffè's test was used for parametrical variables while an improved version of Kruskal Wallis test was applied for non-parametrical variables. The Spearman's rank correlation coefficient was used to test the strength of correlation for non-parametric variables. The Cuzick np trend test, which is an extension to the Kruskal Wallis test, was used to perform the non-parametric test for trend across ordered groups. Real time qRT-PCR normalized values for the genes were dichotomized for subsequent analysis with respect to their median value of expression. Tumors with gene expression levels lower or higher than the median value were classified as low or high status of expression, respectively. The log-rank test was used to evaluate the dependence of patients' survival on genes'characteristics. A Cox regression model was used to confirm the results of the log-rank test.

All p-values are two-sided with values <0.05 regarded as statistically significant. P-values between 0.05 and 0.07 were considered “borderline”.

Statistical analyses were performed with the Stata/SE 9.2 package (Stata, College Station, Tex.).

Results

Case Studies: Clinical and Pathological Features

The total case study was composed of 163 patients with recurrent colorectal cancer. Of these, 93 patients were treated with standard chemotherapy plus Cetuximab, five patients received Panitumumab, whereas 65 patients received only a standard chemotherapy. The total case study included 97 males and 66 females with an average age at the first diagnosis of colorectal cancer of 62.9 years (range 3)-88 years). Forty-four patients were of stage II at initial diagnosis of CRC (33%), 61 were of stage III (38%) and 47 were of stage IV (29%). CRC stages were determined according to the AJCC cancer staging manual. For one case information on initial stage was missing. 54 were proximal tumors and 102 were distal. For seven cases no information on the location of the primary tumor was obtained. Regarding tumor differentiation, 11 specimens were classified as G1, 126 as G2 and 26 as G3. Patients treated with the monoclonal antibodies were followed up from the start of treatment for recurrent disease until cancer progression (PFS) or colorectal cancer specific death (OS) or 30 Apr. 2011, whichever came first. Patients without biological therapy were followed up from the standard chemotherapy administration (mostly based on FOLFIRI regimen) until colorectal cancer specific death (OS) or 31 Aug. 2005.

Clinical details, separately shown for the two groups of patients, are listed in the following table (Table 3).

TABLE 3
Characteristics of colon cancer patients in the two treatment cohorts;
p = level of significance for association.

| | |
|---|---|
| NO | YES |
| biological | biological |
| therapy | therapy |
| N = 65 | N = 98 |

| | | | | | |
|---|---|---|---|---|---|
| VARIABLE | N^{o} | % | N^{o} | % | p |

| | | | |
|---|---|---|---|
| Age, mean (SD), years | 67.3 (10.2) | 61 (8.8) | <0.01 |

| | | | | | |
|---|---|---|---|---|---|
| Sex | | | | | 0.67 |
| Male | 40 | 62% | 57 | 58% | |
| Female | 25 | 38% | 41 | 42% | |
| Tumor location | | | | | 0.45 |
| Proximal | 24 | 38% | 30 | 33% | |
| Distal | 39 | 62% | 63 | 67% | |
| Tumor grade | | | | | 0.10 |
| G1 | 8 | 12% | 3 | 3% | |
| G2 | 47 | 73% | 79 | 81% | |
| G3 | 10 | 15% | 16 | 16% | |
| Tumor stage at first | | | | | <0.01 |
| diagnosis | | | | | |
| II | 41 | 63% | 13 | 14% | |
| III | 18 | 28% | 43 | 44% | |
| IV | 6 | 9% | 41 | 42% | |

All the molecular analyses were performed on tissue samples of the primary colorectal tumors.

KRAS Mutational Analysis

The mutation analysis of KRAS was performed only on tumor samples from the 98 patients treated with the monoclonal antibodies. A mutation in KRAS was found in 33 (33.7%) tumor samples. Of these, 25 caused the single amino acid substitutions in the first or second base of codon 12; 8 were located at the second base of codon 13. Double mutations in the same patient were not found. Details on the mutation types are reported in Table 4.

TABLE 4
Frequency of mutations in KRAS codons 12 and 13 in
colorectal cancer patients.

| Nucleotide | | |
|---|---|---|
| change | Amino acid change | N^{o} of mutated cases and % |
| KRAS | | |
| codon 12 | | |
| G35A | Gly-Asp; G12D | 9 (36% of all codon 12 mutations) |
| G35T | Gly-Val; G12V | 10 (40% of all codon 12 mutations) |
| G35C | Gly-Ala; G12A | 2 (8% of all codon 12 mutations) |
| G34A | Gly-Ser; G12S | 2 (8% of all codon 12 mutations) |
| G34T | Gly-Cys; G12C | 2 (8% of all codon 12 mutations) |
| KRAS | | |
| codon 13 | | |
| G38A | Gly-Asp; G13D | 8 (100% of all codon 13 mutations) |

No statistical significant associations were observed between KRAS G13D mutations and age at diagnosis, tumor stage, tumor location and tumor grade, respectively (p=0.11; p=0.57 and p=0.40 and p=0.20). On the contrary, an association was found between KRAS G13D and sex, with 85% of patients showing the mutation being female (p=0.01). For all the other KRAS mutations, no statistical significant correlations were observed with age at diagnosis, sex, tumor stage, tumor location and tumor grade, respectively (p=0.47; p=0.15; p=0.81; p=0.07 and p=1.0).

Candidate Biomarker Analysis

We studied a polymorphism of the EGFR gene as a new candidate biomarker of Cetuximab and/or Panitumumab therapy efficacy. This polymorphism is located in the EGFR tyrosine kinase domain at nucleotide 2607 of the corresponding EGFR mRNA, codon 787 (Gin), and it changes nucleotide 2607 from G to A, but without amino acid substitution (silent mutation). Three genotypes may be identified: GG, AG and AA.

The candidate biomarker was evaluated at the DNA level in all of the 163 patients of the case study, while the evaluation of candidate biomarker in relation to its mRNA expression levels was performed only in patients receiving biological therapy. The assay to evaluate the alteration of the gene at DNA level was successful in all 163 patients. GG genotype was found in 20 patients ( )%), AG genotype was detected in 67 patients (4)%) and AA genotype was identified in 76 patients (47%). In colorectal cancer patients, no statistical significant correlations were observed between the alterations and clinical-pathological parameters, except for tumor location. AA genotype was associated to distal location (borderline; p=0.06). Alteration types were unrelated to KRAS type of mutations (Table 5).

TABLE 5
Clinical-pathological characteristics of colorectal cancers according
to the “alteration”; p = level of significance for association.

| | GG | | AG | | | | |
|---|---|---|---|---|---|---|---|
| | genotype | | genotype | | AA genotype | | |
| VARIABLE | N^{o} | % | N^{o} | % | N^{o} | % | p |
| Age, mean (SD), years | 64.3 (9.2) | | 62.2 (9.6) | | 63.2 (10.6) | | 6.61 |
| Cetuximab treatment | | | | | | | 0.66 |
| No | 9 | 45% | 24 | 36% | 32 | 42% | |
| Yes | 11 | 55% | 43 | 64% | 44 | 58% | |
| Sex | | | | | | | 0.63 |
| Male | 12 | 60% | 37 | 55% | 48 | 63% | |
| Female | 8 | 40% | 30 | 45% | 28 | 37% | |
| Tumor location | | | | | | | 0.06 |
| Proximal | 8 | 40% | 28 | 44% | 18 | 25% | |
| Distal | 12 | 60% | 36 | 56% | 54 | 75% | |
| Tumor grade | | | | | | | 0.33 |
| G1 | 3 | 15% | 4 | 6% | 4 | 5% | |
| G2 | 16 | 80% | 53 | 79% | 57 | 75% | |
| G3 | 1 | 5% | 10 | 15% | 15 | 20% | |
| Tumor stage at first | | | | | | | 0.46 |
| diagnosis | | | | | | | |
| II | 9 | 45% | 20 | 30% | 25 | 33.% | |
| III | 8 | 40% | 28 | 42% | 25 | 33% | |
| IV | 3 | 15% | 19 | 28% | 25 | 33% | |
| KRAS codon 12 mutations | | | | | | | 0.46 |
| No | 10 | 91% | 31 | 72% | 32 | 73% | |
| Yes | 1 | 9% | 12 | 28% | 12 | 27% | |
| KRAS G13D mutation | | | | | | | 0.63 |
| No | 10 | 91% | 41 | 95% | 40 | 91% | |
| Yes | 1 | 9% | 2 | 5% | 4 | 9% | |

The mRNA levels of the EGFR gene were analyzed by real time PCR in the case study of patients treated with the monoclonal antibodies. The expression levels of this gene were unrelated with age at diagnosis, sex, tumor location, tumor grade, tumor stage, KRAS codon 12 and codon 13 mutations or the different alteration types of the candidate biomarker, respectively (p=0.54, p=0.94; p=0.86; p=0.85; p=0.28; p=0.59; p=0.57 and p=0.31).

Survival Analysis

Among the 163 patients, 115 died because of colorectal cancer at the end of the follow-up period in April 2011. Patients who received monoclonal antibodies in addition to standard therapy had a mean follow up of 14.3 months (25th-75th percentile=7.9-19.2 mo), versus 15.9 months (25th-75th percentile=8.6-25 mo) of those treated with only standard chemotherapy. The effect of clinical and pathological parameters on PFS and OS was studied by log rank test. All these parameters were unrelated to patients' PFS or OS (respectively, p=0.94 and p=0.86 for age at diagnosis; p=0.22 and p=0.99 for sex; p=0.86 and p=0.60 for tumor location; p=0.99 and p=0.07 for tumor grade; p=0.44 and p=0.49 for tumor stage).

Role of KRAS in Cetuximab Treatment

The effect of KRAS mutations on PFS and OS was studied by log rank test in the group of patients treated with biological therapy. Patients with KRAS G13D mutations were excluded from the analysis because it was reported that patients with these mutations behave in a similar way of KRAS wild type patients (De Roock et al., 2010). In our case study, patients having the G13D mutation showed a mean progression free survival of 6.6 months versus the 5.1 months of survival of patients with other KRAS mutations (p=0.18).

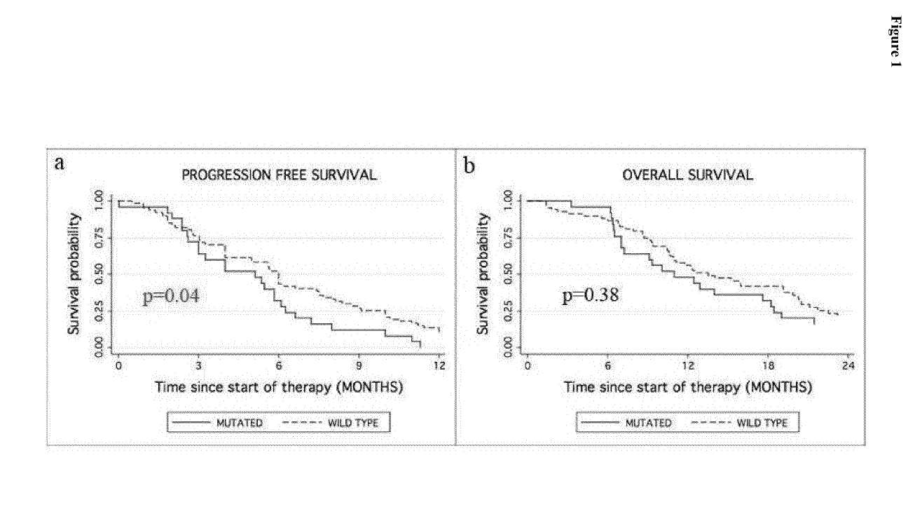

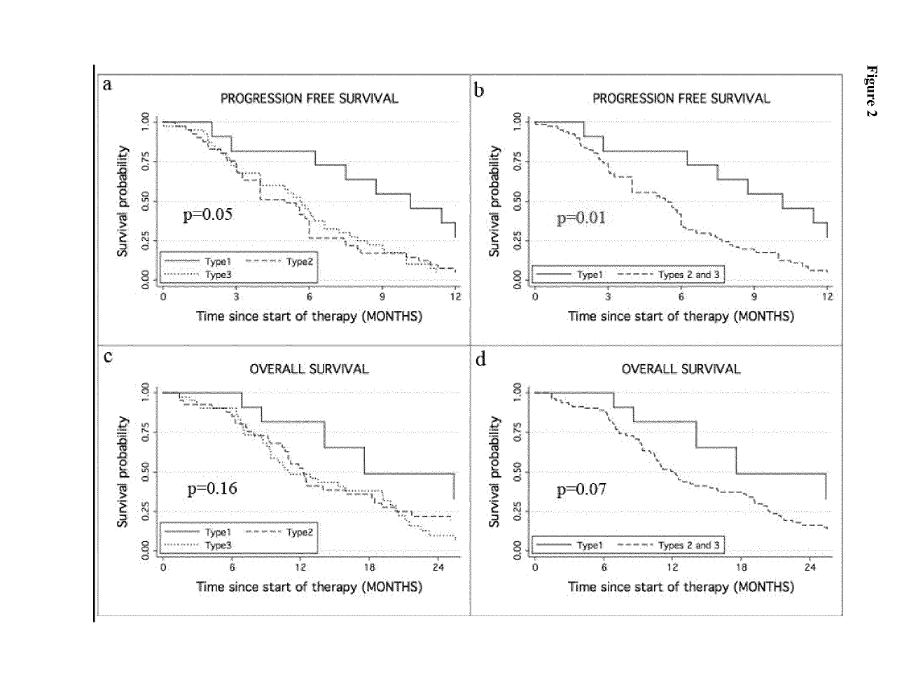

A significant relationship between KRAS codon 12 mutations and PFS was observed after Cetuximab/Panitumumab treatment: patients with a wild type KRAS had a longer PFS (p=0.04) (FIG. 1). No effect on OS was detected (p=0.38) (FIG. 1). In detail, at six months of follow up, survival was 50% for patients displaying wild type KRAS versus 32% of those with a mutation in the gene.

Role of the candidate biomarker evaluated at the DNA level In order to evaluate the role of the candidate biomarker, PFS and OS of Cetuximab/Panitumumab treated patients were studied by log rank tests in reference to the alteration evaluated at the DNA level.

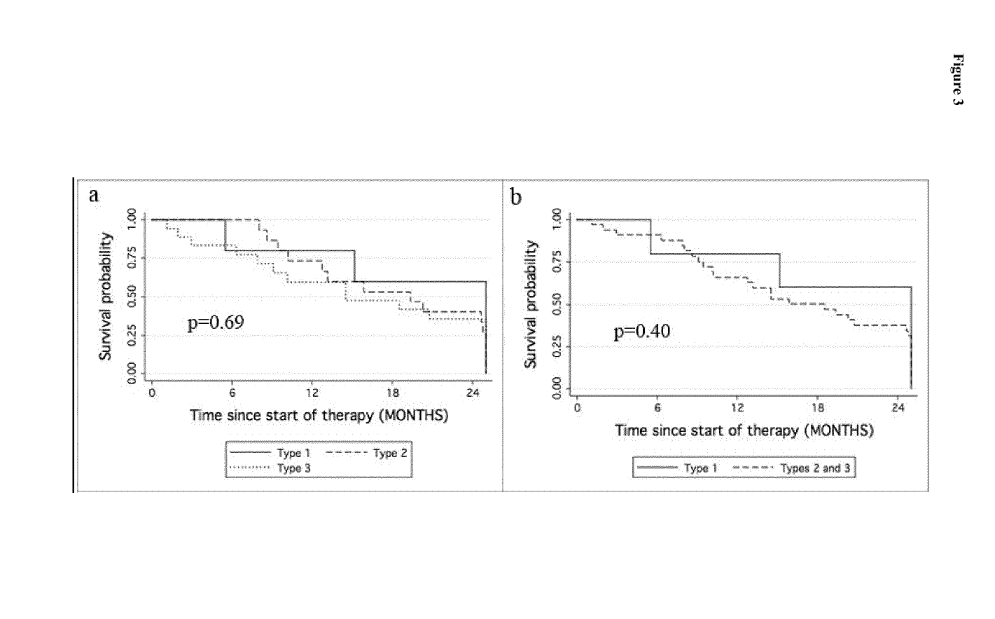

A significant relationship between PFS and the biomarker's alteration types was observed (p=0.05) (FIG. 2a). In particular, patients with GG genotype presented a longer survival than those with AG or AA genotypes (FIGS. 2a, b). Considering that the latter two behave in a similar manner, they were coupled and their joint effect on survival was compared to that of GG genotype. The survival advantage of patients having GG genotype was in this way even more evident (p=0.0) for PFS and p=0.07 for OS) (FIGS. 2b, d). In detail, at 6 months of follow up, after Cetuximab treatment, a survival of 81% can be derived for patients with the GG genotype, versus 34% of patients harbouring AG or AA genotypes. Interestingly, KRAS testing only in patients with AG or AA genotypes cannot identify whose patients have a longer survival (p=0.17 for PFS and p=0.73 for OS).

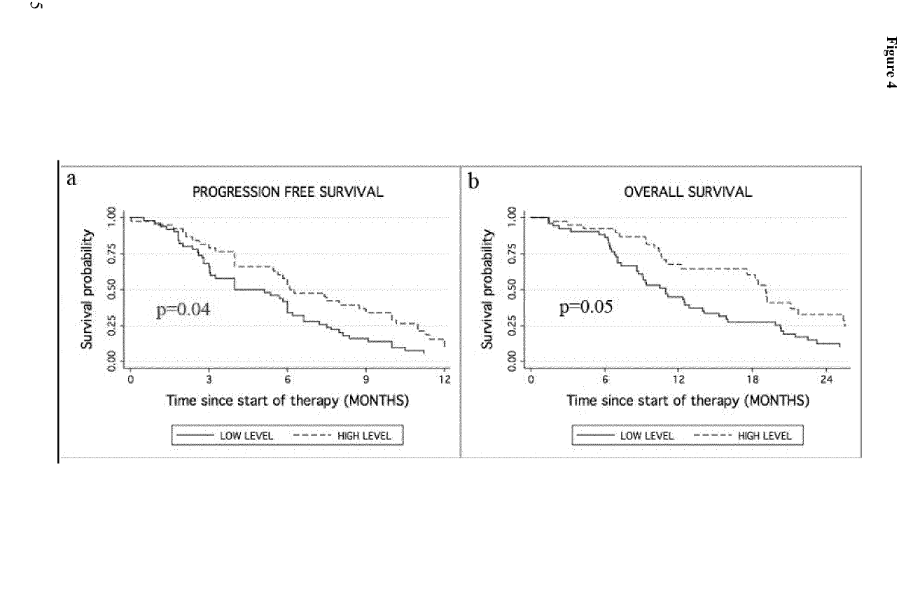

To confirm that the better survival of the patients with GG genotype was dependent on Cetuximab/Panitumumab therapy, we have evaluated the effect on overall survival of the three alterations in the 65 recurrent colorectal cancer patients not treated with the monoclonal antibodies. In this group of untreated patients, our biomarker did not affect patients' overall survival (neither if the three alterations were considered separately, p=0.61, nor if GG genotype was compared to joint AG or AA genotypes, p=0.32) (FIG. 3).

Role of Candidate Biomarker Evaluated at mRNA Level

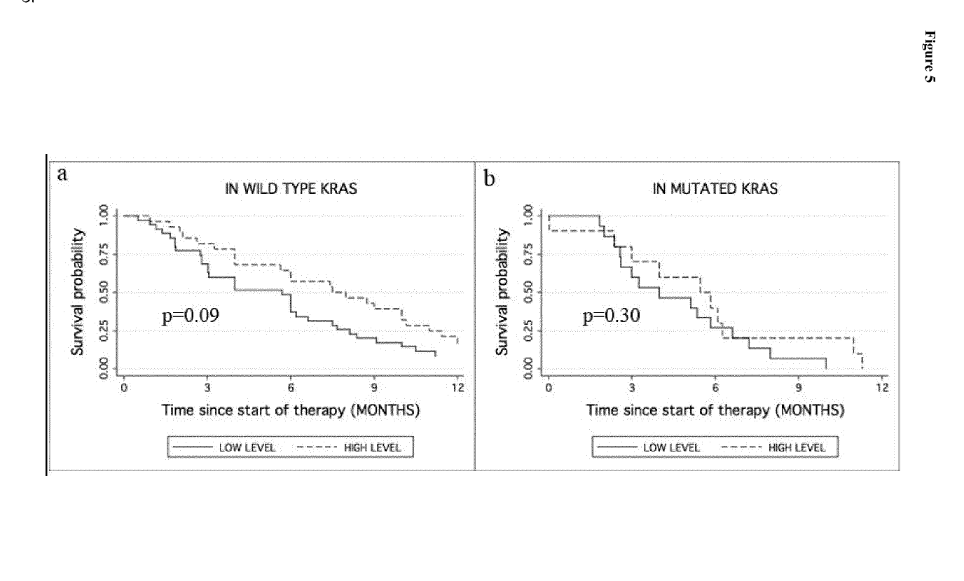

The effect on PFS and OS of the mRNA levels of the EGFR gene was studied by log rank test in monoclonal antibodies-treated patients. It seemed that this gene had an effect on progression free survival. The group of patients with a high expression status of EGFR indeed showed a higher PFS in comparison to those characterized by a low status of EGFR (p=0.04) (FIG. 4). In particular, 53% of patients characterized by high gene levels did not show disease progression within the first 6 months of follow up versus the 34% of those showing low levels of the gene.

After stratifying patients according to KRAS codon 12 mutational status, we observed that the better progression free survival of patients showing higher levels of EGFR was maintained only in patients with wild type KRAS, but not in those with mutated KRAS tumors (p=0.09 and p=0.30) (FIG. 5).

Multivariate Analysis

The significance of the analyzed DNA SNP of the EGFR gene as a predictive marker of response to biologic therapy was confirmed by Cox regression analysis where the contributions of clinical-pathological parameters and KRAS mutational status were taken into consideration (Table 6). The analysis showed that patients with the AG or AA genotypes had almost a 3-fold higher risk of progression after Cetuximab/Panitumumab treatment compared to patients showing GG genotype.

TABLE 6
Results of Cox multivariate analysis for PFS
(Key: ^{a}= confidence interval).

| Variables | Hazard ratio (HR) (p) | 95% CI^{a} |
|---|---|---|
| Age at diagnosis | 1.01 (0.18) | 0.99-1.04 |
| Sex (female-male) | 1.33 (0.19) | 0.87-2.03 |
| Tumor location (distal-proximal) | 1.02 (0.92) | 0.62-1.55 |
| Tumor grade (G3-G2-G1) | 1.11 (0.73) | 0.64-1.91 |
| Tumor stage (IV-III-II) | 0.94 (0.56) | 0.69-1.22 |
| KRAS codon 12 mutations (no-yes) | 0.73 (0.16) | 0.46-1.13 |
| Candidate biomarker at DNA level | 2.70 (<0.01) | 1.32-5.50 |
| (types 2 and 3-type 1) | | |
| Candidate biomarker at mRNA | 0.67 (0.08) | 0.43-1.03 |
| level (high-low) | | |

Considering that those patients with GG genotype benefit from the use of the biological therapy, we investigated the role of the above studied markers only in patients with AG or AA genotypes. Using Cox regression analysis we found that patients having higher levels of expression of the EGFR gene were those with better survival (Table 7). In particular, among patients with AG or AA genotypes, the hazard ratio for the gene was 0.54 meaning that patients with a high level of the EGFR gene had half the risk of progression after biological therapy with respect to those patients showing a low level of this gene.

TABLE 7
Results of Cox multivariate analysis for PFS
(Key: ^{a}= confidence interval).

| Variables | Hazard ratio (HR) (p) | 95% CI^{a} |
|---|---|---|
| Age at diagnosis | 1.02 (0.10) | 0.99-1.05 |
| Sex (female-male) | 1.32 (0.23) | 0.84-2.09 |
| Tumor location (distal-proximal) | 0.87 (0.58) | 0.53-1.43 |
| Tumor grade (G3-G2-G1) | 1.26 (0.36) | 0.76-2.11 |
| Tumor stage (IV-III-II) | 1.03 (0.78) | 0.72-1.27 |
| KRAS codon 12 mutations (no-yes) | 0.77 (0.28) | 0.48-1.23 |
| Candidate biomarker at mRNA level | 0.55 (0.02) | 0.33-0.91 |
| (high-low) | | |

### CONCLUSIONS

Our data confirmed that CRC patients without KRAS mutations in exon 2 have a longer progression free survival than patients carrying mutations.

However, as KRAS mutational status alone cannot completely predict the treatment response to anti-EGFR molecule treatment, a further, biomarker, i.e. the genotype at rs1050171 was evaluated.

Here, it was found that CRC patients exhibiting a specific genotype at rs1050171, i.e. genotype GG show a positive treatment response to treatment with an anti-EGFR molecule, in particular treatment with Cetuximab and/or Panitumumab. Said patients show a longer progression free as well as overall survival upon treatment with anti-EGFR molecules, independent of their sex, age, tumor grade and KRAS mutational status. Hence, the genotype GG at rs1050171 can be used for predicting the treatment response to anti-EGFR molecule treatment in CRC patients. Although in the above examples the correlation between the antibodies Cetuximab and/or Panitumumab commonly used in CRC treatment and the rs1050171 status has been assessed, it is reasonable to assume that the treatment response to any anti-EGFR molecule (in particular Erlotinib and Gefitinib) may be predicted by assessing the rs1050171 status since any anti-EGFR molecule will interfere with the same pathway as said antibodies (see particularly Ciardiello and Tortora (2001)).

For patients habouring one of the two alternative genotypes at rs1050171, i.e. genotype AG or AA it has been shown that those patients exhibiting a high EGFR expression level respond positively to anti-EGFR molecule treatment. Thus, the high EGFR expression level in combination with genotypes AG or AA at rs 1050171 may also be used for predicting the treatment response to anti-EGFR molecule treatment in CRC patients.

### REFERENCES

Choi J E, Park S H, Kim K M, Lee W K, Kam S, Cha S I, Kim C H, Kang Y M, Kim Y C, Han S B, Jung T D, Park J Y (2007) Polymorphisms in the epidermal growth factor receptor gene and the risk of primary lung cancer: a case-control study. BMC cancer 7: 199

Ciardiello F and Tortora G (2001) A Novel Approach in the Treatment of Cancer: Targeting Epidermal Growth Factor Receptor. Clinical Cancer Research 7, 2958-2970

De Roock, W., Jonker, D. J., Di Nicolantonio, F., Sartore-Bianchi, A., Tu, D., Siena, S., Lamba, S., Arena, S., Frattini, M., Piessevaux, H., et al. (2010). Association of KRAS p.G13D mutation with outcome in patients with chemotherapy-refractory metastatic colorectal cancer treated with cetuximab. Jama 304, 1812-1820.

De Roock W. et al., (2010 b) Effects of KRAS, BRAF, NRAS, and PIK3CA mutations on the efficacy of cetuximab plus chemotherapy in chemotherapy-refractory metastatic colorectal cancer: a retrospective consortium analysis, Lancet Oncol 11: 753-62

Di Nicolantonio, F., Martini, M., Molinari, F., Sartore-Bianchi, A., Arena, S., Saletti, P., De Dosso, S., Mazzucchelli, L., Frattini, M., Siena, S., et al. (2008). Wild-type BRAF is required for response to panitumumab or cetuximab in metastatic colorectal cancer. J Clin Oncol 26, 5705-5712.

Donada, M., Bonin, S., Nardon, E., De Pellegrin, A., Decorti, G., and Stanta, G. (2010). Thymidilate synthase expression predicts longer survival in patients with stage II colon cancer treated with 5-flurouracil independently of microsatellite instability. Journal of cancer research and clinical oncology 137, 201-210.

Edge, S. B., D R; Compton, C C; Fritz, A G; Greene, F L; Trotti, A (2010). AJCC cancer staging manual, Seventh edn (New York; Dordrecht; Heidelberg; London, Springer).

Fukushima T, Favereaux A, Huang H, Shimizu T, Yonekawa Y, Nakazato Y, Ohagki H (2006) Genetic alterations in primary glioblastomas in Japan. Journal of neuropathology and experimental neurology 65: 12-8

Kaneko K, Kumekawa Y, Makino R, Nozawa H, Hirayama Y, Kogo M, Konishi K, Katagiri A, Kubota Y, Muramoto T, Kushima M, Ohmori T, Oyama T, Kagawa N, Ohtsu A, Imawari M (2010) EGFR gene alterations as a prognostic biomarker in advanced esophageal squamous cell carcinoma. Front Biosci 15: 65-72

Longatto-Filho A, Pinheiro C, Martinho O, Moreira M A, Ribeiro L F, Queiroz G S, Schmitt F C, Baltazar F, Reis R M (2009) Molecular characterization of EGFR, PDGFRA and VEGFR2 in cervical adenosquamous carcinoma. BMC cancer 9: 212

Marx A H, Zielinski M, Kowitz C M, Dancau A M, Thieltges S, Simon R, Choschzick M, Yekebas E, Kaifi J T, Mirlacher M, Atanackovic D, Brummendorf T H, Fiedler W, Bokemeyer C, Izbicki J R, Sauter G (2010) Homogeneous EGFR amplification defines a subset of aggressive Barrett's adenocarcinomas with poor prognosis. Histopathology, 57(3): 418-26

Nardon, E., Donada, M., Bonin, S., Dotti, I., and Stanta, G. (2009). Higher random oligo concentration improves reverse transcription yield of cDNA from bioptic tissues and quantitative RT-PCR reliability. Experimental and molecular pathology 87, 146-151.

Pugh T J, Bebb G, Barclay L, Sutcliffe M, Fee J, Salski C, O'Connor R, Ho C, Murray N, Melosky B, English J, Vielkind J, Horsman D, Laskin J J, Marra M A (2007) Correlations of EGFR mutations and increases in EGFR and HER2 copy number to gefitinib response in a retrospective analysis of lung cancer patients. BMC cancer 7: 128

Pfaffl, M. W. (2001). A new mathematical model for relative quantification in real-time RT-PCR. Nucleic acids research 29, e45.

Saridaki, Z., Georgoulias, V., and Souglakos, J. (2010). Mechanisms of resistance to anti-EGFR monoclonal antibody treatment in metastatic colorectal cancer. World J Gastroenterol 16, 1177-1187.

Sartore-Bianchi, A., Di Nicolantonio, F., Nichelatti, M., Molinari, F., De Dosso, S., Saletti, P., Martini, M., Cipani, T., Marrapese, G., Mazzucchelli, L., et al. (2009a). Multi-determinants analysis of molecular alterations for predicting clinical benefit to EGFR-targeted monoclonal antibodies in colorectal cancer. PloS one 4, e7287.

Sartore-Bianchi, A., Martini, M., Molinari, F., Veronese, S., Nichelatti, M. Artale, S., Di Nicolantonio, F., Saletti, P., De Dosso, S., Mazzucchelli, L., et al. (2009b). PIK3CA mutations in colorectal cancer are associated with clinical resistance to EGFR-targeted monoclonal antibodies. Cancer research 69, 1851-1857.

Sasaki H, Endo K, Takada M, Kawahara M, Tanaka H, Kitahara N, Matsumura A, Iuchi K, Kawaguchi T, Okuda K, Kawano O, Yukiue H, Yokoyama T, Yano M, Fujii Y (2008) EGFR polymorphism of the kinase domain in Japanese lung cancer. The Journal of surgical research 148: 260-3

Stanta, G. (2011). Guidelines for Molecular Analysis in Archive Tissues, First edn (Berlin Heidelberg, Springer-Verlag GmbH).

Taguchi T, Tsukuda M, Imagawa-Ishiguro Y, Kato Y, Sano D (2008) Involvement of EGFR in the response of squamous cell carcinoma of the head and neck cell lines to gefitinib. Oncology reports 19: 65-71

Tol, J., Nagtegaal, I. D., and Punt, C. J. (2009). BRAF mutation in metastatic colorectal cancer. The New England journal of medicine 361, 98-99.

Van Cutsem, E., Kohne, C. H., Hitre, E., Zaluski, J., Chang Chien, C. R., Makhson, A., D'Haens, G., Pinter, T., Lim, R., Bodoky, G., et al. (2009). Cetuximab and chemotherapy as initial treatment for metastatic colorectal cancer. The New England journal of medicine 360, 1408-1417.

Wu Y L, Zhong W Z, Li L Y, Zhang X T, Zhang L, Zhou C C, Liu W, Jiang B, Mu X L, Lin J Y, Zhou Q, Xu C R, Wang Z, Zhang G C, Mok T (2007) Epidermal growth factor receptor mutations and their correlation with gefitinib therapy in patients with non-small cell lung cancer: a meta-analysis based on updated individual patient data from six medical centers in mainland China. J Thorac Oncol 2: 430-9

Zhang W, Stabile L P, Keohavong P, Romkes M, Grandis J R, Traynor A M, Siegfried J M (2006) Mutation and polymorphism in the EGFR-TK domain associated with lung cancer. J Thorac Oncol 1: 635-47

1. A method for predicting the treatment response to an anti-epidermal growth factor receptor (EGFR) molecule in a patient suffering from colorectal cancer, comprising
a) providing a nucleic acid sample from the patient suffering from colorectal cancer; and
b) performing a single nucleotide polymorphism (SNP) genotyping analysis at rs1050171 on said sample, wherein genotype GG at rs1050171 is indicative for a positive treatment response to an anti-EGFR molecule.

2. The method according to claim 1, further comprising step c) of determining the EGFR expression level if the SNP genotyping analysis shows genotype AG or AA at rs1050171, wherein genotypes AG or AA at rs1050171 in combination with a high EGFR expression level are indicative for a positive treatment response to an anti-EGFR molecule.

3. The method according to claim 2, further comprising step d) of determining the KRAS mutational status, wherein genotypes AG or AA at rs1050171 in combination with a high EGFR expression level and wild-type KRAS status are indicative for a positive treatment response to an anti-EGFR molecule.

4. The method according to claim 1, wherein the anti-EGFR molecule for which a treatment response is to be predicted is selected from the group consisting of anti-EGFR antibodies, small molecules directed to EGFR, and inhibitory polynucleotides capable of interfering with the expression and/or function of EGFR.

5. The method according to claim 4, wherein the anti-EGFR molecule is an anti-EGFR antibody.

6. The method according to claim 5, wherein the anti-EGFR antibody is selected from the group consisting of Cetuximab and Panitumumab.

7. The method according to claim 4, wherein the anti-EGFR molecule is a small molecule directed to EGFR.

8. The method according to claim 7, wherein the small molecule directed to EGFR is selected from the group consisting of Erlotinib and Gefitinib.

9. The method according to claim 1, wherein the colorectal cancer is metastatic colorectal cancer.

10. A method of treating a patient suffering from colorectal cancer, the method comprising: (a) providing a patient suffering from colorectal cancer, wherein the patient exhibits (1) genotype GG at rs1050171 or (2) genotype AG or AA at rs1050171 and a high expression level of EGFR; and (b) administering to the patient provided in step (a) an anti-EGFR molecule.

11. The method according to claim 10, wherein the patient as defined under item (2) exhibits a wild-type KRAS status.

12. The method according to claim 10, wherein the anti-EGFR molecule is selected from the group consisting of anti-EGFR antibodies, small molecules directed to EGFR, and inhibitory polynucleotides capable of interfering with the expression and/or function of EGFR.

13. The method according to claim 12, wherein the anti-EGFR molecule is an anti-EGFR antibody.

14. The method according to claim 13, wherein the anti-EGFR antibody is selected from the group consisting of Cetuximab and Panitumumab.

15. The method according to claim 12, wherein the anti-EGFR molecule is a small molecule directed to EGFR.

16. The method according to claim 15, wherein the small molecule directed to EGFR is selected from the group consisting of Erlotinib and Gefitinib.

17. The method according to claim 10, wherein the colorectal cancer is metastatic colorectal cancer.


SEQUENCE LISTING (19 sequences)

SEQ ID NO: 1 - DNA, 52 bp, Artificial Sequence
  rs1050171
ggcatctgcctcacctccaccgtgcarctcatcacgcagctcatgcccttcg

SEQ ID NO: 2 - DNA, 18 bp, Artificial Sequence
  forward primer exon 20 of the EGFR gene
cacactgacgtgcctctc

SEQ ID NO: 3 - DNA, 20 bp, Artificial Sequence
  reverse primer exon 20 of the EGFR gene
ggatcctggctccttatctc

SEQ ID NO: 4 - DNA, 24 bp, Artificial Sequence
  KRAS first round forward primer
ttaaccttatgtgtgacatgttct

SEQ ID NO: 5 - DNA, 24 bp, Artificial Sequence
  KRAS first round reverse primer
caagatttacctctattgttggat

SEQ ID NO: 6 - DNA, 24 bp, Artificial Sequence
  KRAS second round forward primer
ttaaccttatgtgtgacatgttct

SEQ ID NO: 7 - DNA, 20 bp, Artificial Sequence
  KRAS second round reverse primer
tggatcatattcgtccacaa

SEQ ID NO: 8 - DNA, 21 bp, Artificial Sequence
  GAPDH forward primer
ccctcaacgaccactttgtca

SEQ ID NO: 9 - DNA, 19 bp, Artificial Sequence
  GAPDH reverse primer
ggtccaccaccctgttgct

SEQ ID NO: 10 - DNA, 20 bp, Artificial Sequence
  EGFR forward primer
ggctctggaggaaaagaaag

SEQ ID NO: 11 - DNA, 20 bp, Artificial Sequence
  EGFR reverse primer
tcaaaagtgcccaactgctg

SEQ ID NO: 12 - DNA, 52675 bp, Homo sapiens
caaacagaaagacaaaaaggtgctgggtgagaaaaggagcagagacataaataaaatatccaattttaagggtatagagaggggattcactcaaggaggggagaccatctatctgctttgagaagctgggaaacaaagtcatagggtcaggatggtgcctgactatggatgctctcaaaagctaggcaccaaggatttggactggattcagctggatataagaagttattacagacttggaagcaagattaagtctccgggaaggggagacttaactgggaccagagatcattttcccctataattttaaaggtacttatcatctttaggtactcattaggtacttagcttgtaactctttccactgttcaaatatatacccagtatgcatgtagcctatatggagcaggcacagagtaaatgtttgatgatgataaaagacatgcggaagaaaggttaatttggcaacatcataaaactgaattgagacaaagaaagccaggaggtaggaaagtcaatgaagaagttattccagaaatgtagctgagaaggaaggaatacagaagaggcagatatgggaaaatactcaggaagtataattaaaaggagctgtgactaattttaataaggactgggttaaaaattaagttttcatgtctaaatgttctggaggaccatgatgtcactcaggtaagatggaggaattgagagagggaattcgttagagggaataacatggggaatttggctttggacaggcatttgccatgataacagaatattcatttagaaatggtccaggaaattggtttgatgagaatgaagtgcctgtgaagagagaggactgaagcttgttataatttcattcacttcaggaatatttacagaggacccaaatgtgctaagaactatgaaaacatagaattaaaagaaatgggcctggaataatttacaacctagtaaagcagttatgggaaaacatatttgcaaaaaaggtatacaaagtataatgaaataagtgtccagtaaggataaagtgcagagtaagtgaattaagcagcacccattcatgtgttcaaattcctgccagagtcaaaaggttgtgctgaagtagagtccatgaaagcatcgtagatggctcctcctgctcaagttcccctgctctgcgtcctgctactctggccacaaccgtctggacccagggttgacacacaaacaaacacaataatcttttagccagacataaagaaggccagccaccaatcaggaaaattgtgtcccataaaggcccttcctattgaacagtgaatgacagacatggccagatcttctctcttggaatgctttgaatgttagtcacagagagtgaccactagaagcacagatagcagtagaagctaagactacatgaaaaagcagtggacagatggtgatttatgagaatggcaaaattactagagtcataggcaatggatacttgttaatgaagggatgagcagggccccacagcctgttgctggctcacaagtgcagttgattgctggactgaacagcagctctccgcctgatgatagggttttttaaagtgtccttattgccttaaagtaaatcctcagcatttgcagtgctctgagggtgtcctagcattttataccttttttctaagagcccaggtaacataagggtactcctgttgttctggctttaattctatctgcagaagagggtttcttgtgaaagaaagggtcagtatggtcttttatctgtacagcagataaaaagggtatgtacgtgcacacctttgtacgtggctgccttcccaggacagtctgacagtagagggtagaaacttcagttgtagctgagagcaggcctggaatccccatgcttatactttttatttcctcccccctttcccattgtgatcacaggctacttcagtgtgcttgtccttggagagagcaagggaagggagagccagggagactgttcaagggagccaccaggctcgagaaagaggaacccctgaagacagtagaaagtgcaggtgccaagaatttgaatatctacatcagagtttctcaatgtgcacacagtgaactaccagtttaggatcatttgatttgctaaaaatgaagattactggtctaccttagaccaactgaataaaatatctgggtgaggggcctaggaacttgcatttttggtaggcatggcaggtgattcctaaagcatttacccttgagacctctatgttaaggaaagaaaggtaatgttgcaaggaggtggtgccggcttctaagaaagtacccaggactgaacggcagaaagacctgacataccatatgtataaattgctgtggaagtgaaaaggaaagagaaagtgtctgaggtaaaactggagtgtggggtgcgtggaacaaatggttggatgcagatttgctttacgaatcatgagcctagatgataactgagaccatgtggatggattaggtttctgctaatgccagaatttttataatcagcataaaagtgctatataaagctttcccctcttctatattatagtccttttaagatgtatggaacatcaactataggaagaacatcatattcacagctgtaagaggaaacaagaacttatcatgcacttgatgttgtacaaaataaatctgtgatttatgcttgagtgaccacaaagtagcatacacataagcgcaaattcattcatttaagaattccttgtgtctattatgtacgagataagtatctctgagctgcacggaatgtggcttatcagaaggtgacctaagtttcaaagcagattttgttaagatgaagacagagattgacaggaggtttaagacactctgtctaaagtaaagatttagagtcacagagttcatggattaggatttagaatccacagagggtccacagattcactcattcaacattccataaatatttattgaatgcctttttgtgtcagagactgtcttaggtgctggaaatttagcagtaaatgaaacagaccaaaacccatgccctcatggagcttacattctgatggtagagagacaagaaaacaaaatagatagtgtattattgaaggtgatgagagctctggagaaaaagtaggaaaagagacagatctgggacaagggcgaaattacagtatcaaagatgatctttttagggaagatctccttttaaaaacactttggaacaaagatttaaatgaggtgccagaggggtagcaagtgcatattccctgaggaagacgcctgcctggcattttcaaggaacagccagtaaccaatgtttatctacgtaagtaaggaagggagaacagtaggatgagagttcagagaagagggtaggggatatcaaataatttaaggccatgtaggatttttgagaagaattttgcttttatgtcaagtggaatgagggccactgatgatctgggagtagagtgactatgatccgacatgaagtatactccattttttaactatgtgaacttgtgccaacgttttaacctctaaatctgtttcgtcatttgtaaaacggtaaaaagtattttacctcataaggttgtcgtgatgattaaataagatgatacgataagtgcaaaagatttagcttgtacttaacatagagtaggcacattttctccccttccctgtctttcacttttctcttctgccccttccacctggcgctaggagggggagactggaataaaccttgcagattacagcccgtgtaagagtagaaaggaaaggatgacagttgatgtaaagccttggttaacagacataatagctgggatttaaattcagctttattggtggtttatgatgtggactagaggaatggaactgaaagtctcggaggaggggcgatcctatcaggtacaggcgctgcttttccagccctcaatcctcaagactctcccaagatacatttctaggtagtttatcaacacagactccgggtatgctagcatgtttaattgccccattgtttaatgtcttaactccacgaactttaactgattaatctgtcttctaattaatgtttgaatgactctcctcaggtctaaactaccaaggccatctctacttaaaaacagttgtcttttgtttgtgatttcaggggccctgggtataagcgaagtccctgtttagagaccttgtgatgggttcaaaatatcaagaaagatagcaaaatatcacaagcctcctgacccgagaagattagcgttgaaagggtctgtcgtgtttgtttgggcctggggctaaattcccagcccaagtgctgaggctgataataatcggggcggcgatcagacagccccggtgtgggaaatcgtccgcccggtctccctaagtccccgaagtcgcctcccacttttggtgactgcttgtttatttacatgcagtcaatgatagtaaatggatgcgcgccagtataggccgaccctgagggtggcggggtgctcttcgcagcttctctgtggagaccggtcagcggggcggcgtggccgctcgcggcgtctccctggtggcatccgcacagcccgccgcggtccggtcccgctccgggtcagaattggcggctgcggggacagccttgcggctaggcagggggcgggccgccgcgtgggtccggcagtccctcctcccgccaaggcgccgcccagacccgctctccagccggcccggctcgccaccctagaccgccccagccaccccttcctccgccggcccggcccccgctcctcccccgccggcccggcccggccccctccttctccccgccggcgctcgctgcctccccctcttccctcttcccacaccgccctcagccgctccctctcgtacgcccgtctgaagaagaatcgagcgcggaacgcatcgatagctctgccctctgcggccgcccggccccgaactcatcggtgtgctcggagctcgattttcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggtacggagcggaccacccctcctgggcccctgcccgggtcccgaccctctttgccggcgccgggcggggccggcggcgagtgaatgaattaggggtccccggaggggcgggtggggggcgcgggcgcggggtcggggcgggctgggtgagaggggtctgcaggggggaggcgcgcggacgcggcggcgcggggagtgaggaatgggcggtgcggggctgaggagggtgaggctggaggcggtcgccgctggtgctgcttcctggacggggaaccccttccttcctcctccccgagagccgcggctggaggcttctggggagaaactcgggccgggccggctgcccctcggagcggtggggtgcggtggaggttactcccgcggcgccccggcctcccctccccctctccccgctcccgcacctcttgcctccctttccagcactcggctgcctcggtccagccttccctgctgcatttggcatctctaggacgaaggtataaacttctccctcgagcgcaggctggacggatagtggtccttttccgtgtgtaggggatgtgtgagtaagaggggaggtcacgttttggaagagcataggaaagtgcttagagaccactgtttgaggttattgtgtttggaaaaaaatgcatctgcctccgagttcctgaatgctcccctcccccatgtatgggctgtgacattgctgtggccacaaaggaggaggtggaggtagagatggtggaagaacaggtggccaacaccctacacgtagagcctgtgacctacagtgaaaaggaaaaagttaatcccagatggtctgttttgcttggtcaagttaaacccgaagaaaacccgcagagcagaagcaaggctttttccttgctagttgagtgtagacagcaatagcaaaaatagtacttgaagtttaatttacctgttcttgtcctttcccctatttcttatgtattaccctcatcccctcgtctcttttatactaccctcattttgcagatgtgttctacatctcaagagttattacagtactccaaaacagcacttacatgattttttaaacttacagaggaattgtagcaatccaccagctaaccgcctgaaatagacttaaacatgtgcatctcctttttttttttttttttgagacacagtctcgctctgttgcccaggctggagtgcaatggcgcggtatcggctcactgaaacctccgcctcctgggttcaagcaattctcctgcctcagcctcccgagtagctgggactagtaggtgcacgccaccatgcccagctaatttttgtatttttagtagagacagagtttcatcatgttggtcaggatggtctccatctgctctgttgcccaggctggagtgcagtggcgccgtctcggctcactgcaacctctgcctcctgcattcaagcaattctcctgcctcagcctcccgaataactgggattacaggtgtctgctgccatgcccggctaattttttgtatttttagtagagacgggggtttcaccatgttggtcaggctggtctagaactcctgacctcgtgatctgcccgcctcggcctcccacagtggcatgtgcatcttatagctgaagtctaagccttcttaaatcttgagatccatcaaaacagacaggttttctaattgttatacaatgtatatgttatgtttataatagaaatcattttacaaataagttataaatgggaaaggtctatttgtaattatcagctcagaattaaccataaaactggtgtcactgaagtgactgaggtccaaaatgctgactctgcatgttatagactacagatatcaaatatggttgctaacaatagtttactttgagactgtagccatccacagtatatttgcttttaagagatggtagatggtaattcagttttatgaaaaataaaaatgaattttcttccattacaaaattgttggattcgagtccagtccactccttactagcttttctaactctcggtgagggatcccctcccagcccatgatcttcatttggtaagactcctttggaacccagttctctctagtggatttaaatgtgatttggttttaaaaatctcattcaaggaattttttttttttctggaaacaaccaccgcataaacaagtaaaccggaagatacatgtggctctgaattcatatatatacacaaactctaatccaatgtctgtccacagtatttcctaggctagtaaactttttggccttaacgacccctctaccctctttgtttttttgagagagagagtctcactctgtcacccaggccggaatgcagtggcgcgatctcggcccgctactacctccgactctcaggctcaagcgattctcccgcctcagcttcccgagtagccgggattacaggctcccgccaccgggctaattgtatttttagatacgggatttcaccatgttggccaggctggtctcgacctcctgacctcaggtgatccgcccgcctaagcctcccaaagtgctgggattacaggccaccacacccggcctacactcttaaaaattatcgaaggggccgggcacattggctcttatctgtaatcccagcactttgggagactgaggcgggaggatcgcttgaggccaggagttggagaccagcgtactcaacatagtgagaccttgttataaagaaaaaaaaaatccaggattaaaaaaaatctttgatttgtttgggatttattaatatttaccgtattggaaattaaaacaattttttaaaatgtattcatttaaaaataataagcccattacttggtaacatgaataaaatattttatgaaaaataactattttccaaaacaaaaccaaaacttagaaaagtggtattgtttcacacttcagtaaatctctttaatgatgtggcttaatagaagatatggattcttatatctgcatctgcattcaatctattatgatcacacatctggaaaacttgtgaaagaatgggagttaaaagggtaaaggacatcttaatgttattatgaaaacagttttgacctcttgcacaccagaaaagtcttagtaacctgaggggttcctagaccacattttgagaactgttttaggctatgcaaactggttggggggaggttggggtaggcagagagctagaagatacattttagtgtaattctcctcatctattcctaattgctttggcctacatttgaaataaagcgtggaggcaaacgggataagatacatgtttgtagtggttgttaacttcaccctagacaagcagccaataagtctaggtagagcagagtaaggcggggaactatgccgtgaccgtgtgtgatacaatttttctagcctgtggtgctttttgcggcagggcttaggagtaaggttagtatgttatcatttgggaaaccaaattattattttgggtcttcagtcaattatgatgctgtgtatatttagtgtttatctacaatatatgcacattcattaatttggagctactcatcctataataaatagttgtgcatttactcccatttttttctgcatttctctccttatttataattatgtgttacatgagggaaaggaggtgaaattaaacattcatattatttcaaaaaatttgaaacaactaactaaaaaatatgttttattttctgtatggtgtttgttatacaatctgtcaatattcatgcacctcttgggagacagtgtatgaaaagcaaagagtaacagtcacatggattactgattactgagatatattcacttgcatctttttttttttttgagacggagtggctctgtcgcccaggctggagtgcagtggcgtgatctcggctcactgcaagctccgcctcctgggttcacgccattcttctgcctcagcctcccaagtagctgggactacaggcgcccgccaccacgcccggctaatttttttatatttttagtagagacggggtttcaccgggttagccaggatggtcttgatctcctgacctcgtgatccaccctcctcggcctcccaaagtgctaggattataggcgtgagccaccgtgcccggctcacttgcatctcttaacagctgttttcttactaaaaacagtgtttatctctaatctttttgtttgtttgtttgttttgagatggagtcttactccgtcacccaatctggagtgcagtggcgtgatctgggctcactgcaacctctgcctcccgggttcaagtgattctccttcctcagcctccccagtagctaggactacaggagagcgccaccacgcctgattaatttttgtatttttagtagagagagggtttcaccatattggccaggctggtcttgaactcctggcctcaggtgatccacccgccttggcctctgaaagtgctgggattacaggcatgagccgccgcacccggctttctaatctttatctttttttgtgcagcggtgatacaggattatgtattgtactgaacagttaattcggagttctcttggtttttagctttattttccccagagatttttttttttttttttttttttgagacggagtcttgctctatcgccaggctggagtgcagtggcgccatctcggctcattgcaacctcggactcctattttccccagagatatttcacacattaaaatgtcgtcaaatattgttcttctttgcctcagtgtttaaatttttatttccccatgacacaatccagctttatttgacactcattctctcaactctcatctgattcttactgttaatatttatccaagagaactactgccatgatgctttaaaagtttttctgtagctgttgcatattgacttctaacacttagaggtgggggtccactaggaaaactgtaacaataagagtggagatagctgtcagcaacttttgtgagggtgtgctacagggtgtagagcactgtgaagtctctacatgagtgaagtcatgatatgatcctttgagagcctttagccgccgcagaacagcagtctggctatttagatagaacaacttgattttaagataaaagaactgtctatgtagcatttatgcatttttcttaagcgtcgatggaggagtttgtaaatgaagtacagttcattacgatacacgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagtacttattacaagataattatgctgaaagttaagttatctgaaatgtaccttgggtttcaagttatatgtaaccattaatatgggaactttactttccttgggagtatgtcagggtccatgatgttcactctctgtgcattttgattggaagtgtatttcagagtttcgtgagagggtagaaatttgtatcctatctggacctaaaagacaatctttttattgtaacttttatttttatgggtttcttggtattgtgacatcatatgtaaaggttagatttaattgtactagtgaaatataattgtttgatggttgatttttttaaacttcatcagcagtattttcctatcttcttctcaacattagagaacctacaactaccggataaattttacaaaatgaattatttgcctaaggtgtggtttatataaaggtactattaccaactttacctttgctttgttgtcatttttaaatttactcaaggaaatactaggatttaaaaaaaaattccttgagtaaatttaaattgttatcatgtttttgaggattattttcagatttttttagtttaatgaaaatttaccaaagtaaagaccagcagcagaatgataagtaaagacctgtaagacaccttgaaggtcatggagtagaacttccatcccaagcagatgaggatttatttaatctcaaagacctccaggaggggacattccccaactgtccttgttaactcattttcagaacatatttattagcatattttacatgtaatttggatcttcatgttaaatttaacatcagtggagatggaaaataagcatatcgccttgtctttgaaatagccctatattgttagattgtttcttaggcttctttaccctgggttaagcagtcctaatactttagcatttattctacatctagtgtactaatttaaaaaaatcagttctgaaaaatttctaagaactttcttcaagttccaagctgtgaaatctagaacaggtcaaagtgccttattaacgtactgtactgtgtagtgtcttgaagagacactttgcgctgaggcaagttctgagggcattgggtggccttgggaagatatttatgcagtttagaacctggagaattgattagataactaatcataaggaaacgtcacatatttttggtactataaaaaagtggagaaataatgcctatttgcaaagatttgatttaaacatagaaacaactttatttggcttccaattttaagaatttacagcagtaaaggggaacagtctaattgaagtagactgcctatgcaatagtctctgtatatttacttttgacaagttaattcaatgtgtactatagttttgtttctttgaagaggtttgaatagtgcacccattttaatctgtattgcaaattcagggttacttggcagactctactatttaaatcagatgtaaaaggaagttttaatataattcactttatgcctgaaagttttcctgggattttggaaggtgattttactggaaatgctgtctgtcttccctgaaaatctgagaaattccattacactttgtttccaatcagaggtcatgagtgctatatgagtatatacagcatgacgtcatgaatgtgataaagtgggttaggaaaccttttgctaatgattgttaaaatgcaatataaatgttgaagaaataaagctaacagttaagcctttatttgggcggaaggctgaaaaagtttataaacttaaacctataactctgcttatgatttctgccaaaccagaagacttgactctgggaagcattggttacctgtgaactttgaaactgacggtccctgacgtagtttagtcacctgggaaaaggtatctgagattatctcttatctcccaagttacagtgagtctctgagggaactgacacattacattaagttcttggtgtagttaaactgtaagaaaggcaggagaacttagtagttaaatagttggttaaatggaaatgctgactccatgttattgtaaaaagttaaaaatttaggaggatatggggatttcactgccattgcaggttttgattggtatttaccaatccgtgtgggtcagagagaaaattagaaaggatatgactgcacattttggaattattagcagtttttctacatttaaaatggaaataaattttttaaaaatttaaatcaagtaatactgtattttttggtgatttagatttttcaaaatttacactaagagatagtaaggagggtggctattgtttctttcaataatgtctctgagaggttgtaactcatctaaggatacgtagctaataagtggtaggatttcaatttaaattctctgagaccaagttaagtagaatttgcactgtactcttgtataactttttaaaactgaaaattagctatctttcaaattaagaaaatatttactaatggagactaattcagatttgtaagtataccaaaatttgaacttagcctgctatctaatggcaacttagtggcagaggtatgatgtaaaatcattcaggtatgacacatagatggagtatgtttgtattcgaggctgtgcacataatcacctttacttgtattgtgaagtatatattgttatcttttatgaagcccactaaagagataatgaaatacctcgttattagggcaagattattgaaaactcaaaatagcccccaaacacaatacttggctagaaatatatacctttatagttcagagatcatttattatcaaaaccctgaagttttttttctaaggtaaaatttggtggaagaggaaaagtctcgttttaaaaaaatgtaggtagttacagagatcagaatgattagttgatcacttaccaaatatatattaagtatctactgtatataatatgctagtaagaataaatatagcaggaagtattttttcccaggctctaattgtttgacatcagcatgcttttattgtggcacttataattcagttcaagtattatgcccctctttgatggaacagtttcctattcagtaaggaagaccagattaatcattggattggtttgtttcatctttagtgttctgagctgtagagtatttatttaccaaggtttattttaatttttattttatttttatttttccatgttcattgtagaattcattttacctacgaatgaagtatgtagattatagagagaaaatttgtaaaattaaactgatactgaagactggtataagaaaagccttatgtaatttgtaagctgctattcttctgagtttatacatatatctttagtaatcaatgagggatggttgggtgactgccctccaggggacatttggcaacatctggagatgtttttggttgccacaacttggggagagagtactgctactggcatctattgagtagatgctattactttaaatggcaaagctgcagttacctttgcaccaacctaatattaaacttcctgcagtgcacgggaaagcccccacaacagggttatctgaccccaaacctcaatggtgttaagatccaaaccttgatatgttaacctgtagctttaaacatcctttaaattgtcaaattcatgtccctgacataaggtttatgttagattttcaagtataacaaagatttaaactttaacttttgtacgttaatgatatgttagcttactccagtcttctattaaaacattctgtttttaaaatcagagacacacagcaattttataaatcatttctcttcaaggctgtgaagctctccccacttttgtgagtgccctctactggtcaaattatttgctttataacaagtaacagtgaaatcctaagtttgtgtagtttcgctgtttaaattatgggtggcatcaatttataaatatattcgttttatttaaaagtcttatatgattgatttcgtatcatttttgctctctgctaatattaatataaagattactgtctgtattagttaggcctaactaagtaggtgagtatagtgaactaagaaaggaaacgaggcagtatataagaaaatagggtggttcagttgttaacacttactgagcttactttgttgaagggactaaaaggcagcagtgtggctctctgagcttctttgcatgcactcaggagctgcttaatggagtccaaggcttggtggtgtgttacaggggatgataggagggtcctattcagaagtggcaaattgtgaaagtgcacattttgtagagttttataggactgtagaatagttgtgagcacctgatttttagaataaacagaaaactcaggtactgtatttaggtcaaattaagaataagtatttattaagacctgaatataaaactttactggtcatggtttttttctaccttgggtttttataaatccaaagatttaaaaacatacaaatggaagttggtaatggaattaagtgaaaggaaaaaatgattttatggtttggaatctcctaagattctggttttaacaatacaactaattccttaatcctagaaatgttcttcactgcccactttgtaccatgcagtcttcctgtgggctagagatacactgaggcgcaaaacagaccagattcctgccttcatggagcttattagttttaggtatctctagatttcttgtaatacctattacaatgcctgcacatcagttcattcatgtgggttcaacgtagtactcagtacatggcaaattcaagttttacttttcggaacttcatggatttttttcctcagaatatcttttatccataattggttgaatctgtagatgcagtacccatggatatggatggcccactttattttgaagagcagtgtttctaggcaatcatgctaattatatatgacttaatttagaggctttatacttaagagcattacatttctggcgtctcttaaccattattatttcataatgtgtaggttatggaacagttaaattattgggatcttaatatagaaattagtagaaataagccagatatggtggctcatgcctgtaatcttagcactttgggaggctgaggctattcgctgtactattttttactacttttctataggtttgaaattttttcaaaataaaacattgaaaaaagtaaggtaggtagtgtgtccctccttaatcctttcaaatattttattttcactatttctattaatttttttttttgtttttgagatggagtctcgctctgttgcccaggctggagtgcagtggcgcgatcttggctcactgcagcctccacctcctgggttccagccattctcctgcctcagcctcctgggtagctggtattacaggcatgcaccaccacacccaattactttttgtatttttagtagagacggggtttcaccatgttggccaggctagtctcgaactcctgacctcgtgatctgcccgcctcagcagtgtcactgcttctagaccgttttcaaggcacagagcttagaaatgcatgttactaagaaatcaagagttaactatttttcaccttctttctcccgcagtgagaaccctggttctaccctgtttctccttgtgtaaattttaatgctaaactatacacttgtgaaataaaaatgataatgtcattcttaaattatggatcttgcagtgttatctaagtaacatagattgagtgatttaactttaggtttccttatttgtggaatttggataaatatttttcacccttgagaaaagtgagactcctttctcatcatcagagtatccttaaaccattaaggcaaacatttgggaaaaaactgagctatctggctgcataaaaattaagttttctttaacaaagatagaagacaaatgaaaacctagaaaaaccatttggttcaagtaacaggaagctatcttatatatgaattagagaaaagcaaacacacaaatagaaaaaaagggatggggggtactaaagatataaatagcttgtctaccaaaaaagaaataaaataaataacatgaacatataaaaagacacttacttcatgaatgtgatgcaagttcaaacaataaataacatttctgtactttcatattggctaaggttaaaatgataactgctaggaagggtatggagaagtgtgcgccttgcactgtagtgggagtatagaccctcagactttatggaggtcagtctggaaatatgtttcaaaatgtaaactacatgtcctttgaccaggtaattcaacttcttgaaatttatccaaggatttaattggataaatgtttaagatgtatatataagaatgtttactgcagtgttgtttatgattttaaaaaaatggaaatcatcttcatgtctaccaatagagaatgggtgaataaattatggtatgtccatatatacaaattacatagttgttggaaatattaggtagatttagatatactgatgttcaaaaatgtccattatgtaagtgaagctgggtcacagcaccttgtgttgagtatgatttcatctagaaacaaaattactccctcatcctttgttgtgttttagttttttaaaataagcttataccattgggctgggggaaaagtaaatactcgttttggagagagaaaagggcactaaagtttcagataccgttagattatttcatgcttatttttcaagcctcaataaattacataattcacatgtagtcttggattaaggaaattgctattaaggctaaataaataatatgagaggtatataatataaaatatgaacattatattggcattaagattggatccacggtcattccagcctctcattcttacctggacttcaagtgatcacttgtgggcaaatgccatctgacttgaacaggttacacatgtatgctcattatatcgttattttcaaaatttgtcatataaattttccttgagttcattcagatttttgaactagttttttctcttgggagtagtacacacttaattctctctagtactaagctaatgttcaccattcttataattttaagtatccagcatttagtaaagaagtctttgttttctttatccttacttttagtgaatgtcttagtttttaattgaaaattctgccatgaaaataagctctttaacatcttcactccctaatcaaaacagaaatccttcatagccttcagttgtagctatccttccctgtgatttgtccagctccattatatttattttgaaatatggtgaccagttttgcaaaattatttcaactgtaggtgcccagtgattttgtaaggagaagatactgtttctgaacagttctcagtagccagtggcctgcccctactttttggcctgcgtgtagtatataaaataatgcagttaactttttatagcacttttcattttataaagagattttcatggtctttaatattaatctatgtataaagtcctgtatgcagttttacctactttcacagctgaaggaacaatagcttagagaagatgtgagataaagtagtttgcccaagcccatagcacaaataagtgaagttcttcggctgtccatggatcgaagactcccaagtctatctctagcctggacttctgtcctgagcaccagacatgtatgtatatcaagatgcctgcaggtcatatccaccaggacaacccatgagtacagggaattcaacatgcccaatatcactcatcttttccttcgccctcccctttgtactcatcccctgtcggtaagctctgttattttaaaaaattgaaatgtattcacatagcatacaatttacacttttcaagtgtacatggtttttagtatattcacaagggttgtgcagtcattactactaattccagaatgttattatcaccccaaaagtcccacatccattagcagccactccccaatcccttctcccaccagcctctaaaaactgctaatttttccatctctgtggatttgtccactctgattatttcatataaagagaatcgtacagacgtggccttttgtgtctggcatcctccacacaggatgatattttcagagttcgtctatgtttttgcttgttgatcattccttcattcctttttctggctgaataatactctgttatatggatataccttattttgtttatctgttcatttgatgggcatttgagtgatttcctctttttggcaattttgaataatgccactataaacatttatgtacacgtttttgtgtgaccatatgttttcacttctctcgggtgtatatctaaggtacagttgctgggttatatggtagctctgtctttgactttttgaggaactgccaagtggttttggtagtgattgtactgtttacattcctaccaacaattttacctaagtatttctcaaatctatttaatcttttcggtccatactgctgttgctgccttagttcagattttgtcatttcttgtaataattcgtagctcatctcccagtctctgctcccctctctccctccctcccccttcttctctctcttatttccacccatttttaacatttatagaagtcaaaagtctagttcagaaagcagaaaccatactagatatttcagcacagagaactaattaggtgttggaagactgaaaggcaaaaaaacactgaagtaacacagtaacatcaagaatgggcactactcctaagattcagggaatgctgggaagatttggggtttatcagaactggaagctcagaggaggggccccttgtcgctgaggcttaatccctgcagaggtgcctttggctgctactggtgaatctgagtgggtatgatgagtcagtgtctgggaagggccaaaacattttgtccctttctataatttgtcatgataatgctagtaatgaatctgatctcccttcctattttaaaaaccttttagtgattttgtataggatgaagtttaaaactccttacttaatatacacatgaccctccgtaagctggcccctgcttgattgtccagtttcacttcttggtgcttattctaaggcctctaagccttagagatcctctaagcctttgagatccccaaaccctggactgcggactggtacccacctgtgtggcctgtgaggaactgggctgcacagccggaaggaggtgagcattacttgccttagctcctgtcagatcggcagcattagattctaataggagcgtgaaccgtgttgtgaactgcccatgcaaggatctaggttgcatactccttaggagaatctaactaatgcttgatggtctgaggtgaaacagtttcatcctgaaatcacccccaactcggtccttggaaaaattgtcttccacgaaactggtccctgatgccggaaaagttggggaccgctgttctaagctaaagttatatggagctccttggttctgtgtcctcaacatgctgttctatgttttttacattctgtttgctccttcctgcttggaatgtccttcccctccccgtctttcttaatgcatacaaagttgatctctcctgtgtgccaccattgtacttcgtcttgcatatggtgttacattcattttattttaattatttatttacgttcatgtctcttccactcaccttagttgcttgaggtcagaaactatataatgtgtgacacggaatgtgacacctagattttcaataagtgtttctatgatacaagggagactgatgtgggtagatgggaatgaactcatcaacctctgtttacataccctaaattccctgtttcttccctattataattctgacagtctacaaccgtctttgatggcttataaacggaaagtgcggaacacatcattctacagtgaatttaaataacctttcggaagagtaacgtaaagtacttgagcattaattgagtaaaagtttctcatcttttcctacaggtgttattaagcagtatgtaaaaagtccttacaatacttaatacattaagaaaacatacaatttcaagaggaaatccccgagtaatacattattgacattttcagcagttctagttatattgagaagagcatctcatggaattggcagaatgaagatggagattaaatgagatgatgtttgtaatatgcttatgacagtatctggcatataagtaagggctcagtaaatgttgactgctgtaattactattaatagtaatatgattacctttagtaaaagttattagtttctttaggttttttgtttactacaatatagtaaacaaaatctatacttggaatgtatatattgttttgttttgatacatggaatatgtctctgtgtcagagtcactgcctgagttggaaaacccatactcgagtatgttaaaaggtgaacacactgaataatttagttattaattataatggaaaaatgacaaacttgatgttctggttaatgaggttatcttatcttgaatgagttagcttttaaattcctcaaaataaaggcatttaataaaccaggaaacacttcattaaaaaaattatgcaagtcagtgtaaaagaagattaaaattccacatgggcaaaggacacacgttggcgataaatatgcagataagaaaaaaaacctatataacattattactcctcaaagaaattggtatgaaaacaataaaaatgtgtagcttatcaaaccaacaaaaatttaaaaatatgaaatccattttaagtaatgataaaatgggtgcactcttagtgctttatagaatagtagtataatgaacctcatgtgtgtaccaaccagctctttcatatcttaacatttagcaacatttgatttagctctttcttttttccaagatagaaaagttaatattgttgaagactcctgcattcttttccctagtcttattttcttccctcccataaatgtgttaaaatctctgtgtgtattgttttggttgtatttttacataaaactttacatattatataaaatttaattgaaggtaaaatttattaaattattcttaatatatattgtaatttaaaaattaacagcttcattgtcttgataaaatttatggtatcttaaacatgtgcttgtttttctaagagaacattgaaacatagattttaaaacaaattgttgaaagattaaaaaatctgcctttgcacactgttacattgaaagtggggcatttgtcgtgaacattcatttcaaatatgtagtatcttcagaatatttgagaaggatttgtattatataattgaaaaatctgttaaattgtatttatgttaactgcttaattctaataaaatttccattcattttttagtatctgcatatatttacatcaaatggattcattcacttatttaagaggcagtactaattacctatagcgttcaagactgttaggtagagggtgtgtagtggtgagtacaacaggcgtgagccctaccaacacggagtttaaagcctagtagaggatatagacttaaacaatttcacaagtaaatacataattacaaattataatacatgctatgaaggaaacataggaggtaccagagaaggaagagtgctttgcatttttatttttaagaccgaagagtgctattggaggactttgagcaagtgaatgacatgatctaacctaccttcgttcattcattcattcattcattttcttccttcctggctcaagcagtcctcccacctgagctccccaaatagctgggactacaggtacacactaccacacctaatttttttttgtattttttgtatttttgatgggattttaccatgttggccaggctggtcttgaactcttgacctcaggtgatccacctgtctcggcctcccaaggtgttgggattataggtgcctagcccatggtgcctagccctaacctacatttataaactatcacttgctgctgtgtggagactatattgtgagattaacagcagggatacctgctaggaagcaattgctgcagattgcctgagacaaaatagttatcatggactagggggatggtggtggtggtggtggtaggtggttggatgtaggatatattttgaagataggtaaatggtgcaagattatgggtcagttttaaatgcttaagtaaattttctttgtaagacattttaggatgccatgttaagaatctctttataactgtcatttaaaaaaaaaccacatattttcttagcataatttcccatagtaacattactatgtcaaaggctatgaacatttgaatgactttagataaatactgtaattgctttccaaaaatattgtgcttattatgtcaccagaaatgtttgaattctgtctacaattcagtcttgccagtatagtacatttcatttagaaaaattttttactatgtagatggaaaaaataatattttagctgggagtggggggactatggggaataactttccttcatttaatattttattgtgagttagtttaagttactttattttatcgtagtttcctaaggctacaaattagtaaccttggtaacttatgtacctaatttaaaagtttacttttttgaaaggctggaaatactaattaaaaacgtaacaccttcatccttgtctttgctccattattaactagtttcattacagaatctctgtgttttaaaatcagatgggttttcataaccagtactttctcagagtggtaaatttaaaaaaatatataaagagaataaataatatttgttgagaatacttcaaataatgtgaagagttattaacttacagcaggagttggcaaacttttctataaagggccatatgggtctttgtcacaaagtcttgggtttttgtttttgtttttttaaacagctatttaactattcctagctaatgggcaatacaaaaacagtgggcaagatttggcctgtgggcagtagcttgctgaaaccttatttagactctaaattttttgaaagagtctacattgatgcatatttttttttcttcctccaaatacagttgacccttgaacaacatgcgtttgagtgaccatgggtccacttgtgatacacgtttttttcccaaccaaatgcagatatggagggctgacttttcatatacctggatgttcctgggccaactgtaggactagaggctgggggggtcttggaaccaatgccgtgtgtataccagggatgactgtttcttatggcctgacctgaagttggaacagaatctttattaatatataatttttgttgcgtttgttttctctttatatttatccattctttttagatcgtatttcatttaacactttttcttctttagtttttaccaagttgcactgaaaatagctcagtgactaattgcacttctaagagtgaggaccctagttaaaattaactctaaaaatactgaatttttaacctaaaccttttatttctaatcaacagtattatttatgagtaggttatagattactttgaaacggaatgtgtctcagaactttgctatcgatatttttaaggtctggtagggaaaagataataggaatgagatttatcagtgaataggggactgctttcccagtttctcggtcgcactggtgtattcaccatggaagcatcttatgaaatatgtacataaactactaatatcccacattacaggttgactattctttatctgaaatgcttaggacctagaagtatttttggattttggtttttcagagtagggatactcagcctacattggtaagtaaagaatgtgaggtgacaggctgggcgcgatggttgacgcctgtaatcccagcactttgggaggccgaggcggatcacctgaggtcaggagttgaagaccagcctggccaatctgtactaaaaatacaaaaattagctggacacagtggcacgtgccagtagtcccagctactcaggaggctgaggtaggagaatcgcttgaacctgggaggcggaggttgcagtgactcgagatcgtgtcactgccctccagcctaggcaacagagcaagactccatctcaaaaaaaaaaaaaaaaaaaaaaaaaaagaatgtgaggtggcagcaataggtaggaagagtctttggtcagctttacatgctctgtagccatgcctgggtaatgggttgactctaagactctgtgctttgctcccacctcctgctttttcattactctttagaatggtttttaatttgtgatctataggagttctttcaagtatttaataagagaataggctaaattaagtaaatgtcaactgaatgctcaaatctctactaaagagcctcttatttagaaaataaatatccatcttttttttctgactggtgagataattaatttttattacagatggtttggaaaataccatatgctttaaaagataagcacaaaattatagtctaatatgtaggttttcatactttaaaaaattgaaaaccaaagaaaaacatttaacatagcatctagtacaaagaaaagagataagcaagagataaatgtcttttttgggacagagttttgctgttgttgcccaggctggagtgcaatggcacaatctcagctcaccgtaacctccacctcccgggttcaagtgattctcctgcctcagcctcccgagtagctgggattacagtcatgcaccaccaggcccaggtaattttgtatgtttagtagagatggggtttctccgtgttggtcaggctgatctcaaactcccgacctcaggtgatctgcccaccttggcctcccaaagtgctgggattacagacatgagccatcgcacccggccaagataaatgtcttttaaattatctccattaaagacataacctttataacattttgatgtatatattaccagtttttaaacacatagtagatttgtataaatacataaacacatattattgtgatcatgctgcacttagacatctttatattctccttatactgtaaacattttgaaatactttactaacaacatttgtaatgaccattctttctctctttctccctctgatagaatggtctacagagtaattcataaactaaacatactttagaggctgggcgcagtggctcatgcctgtaatcccagcactttgagaggctgaggcgtgcagatcacgaggtcaggagttagagaccagcctgactaacatggtgaaaccccatctctactaaaaaaacagtacaaaaattagccgggcgtggtggcgtgcacctagaatcccagctactcaagaggctgaggcaggagaatcactcgagcccaggaggcagaggttgtagtgagccgagattgcaccacagcactccagcctgggcgacagagcgagactccatctcaaaaaaaaaaaaaaaagatacattaatactatagcctacatgtggaacattaagaaaataattgcttttatgtttatgctttatacctgttgttagccctgcttcttatttcatgatttcatggcttcacattgtaacatccctttaccatattttttgaggactgttttggcagaatgtgtgaaatcttgagcagaagtattacccaaaagtcagaagaaaatcagatttttatttcaagattctgttaaagttacccactcccttcttttacttaatcttatagttgcagttctctctctttttagaaaagaaaaaagaggcccctcaggatttgcagatgaaacaatattgctctttagagatatccatctggctgttagattatttttccacagttttcagaagtggatgaggccattagaatcttgagtattgcccatttccttatgtgtgcctttgactatagataaaatagatgcatgacaattatttataagttgattgatttttcttgtcatttaaatcatcttgaataatagagttggtagagctatcccatttttgaaattattttgttttgtcaataactttttgttaccagcatgtacacttgcattgttgactctccatataatacctttaaaaaatttttttttgtggtaaaatatgcataacataaagtttaccatggtagttttctttcatttgttttgtttttgtttttttgagacggagccttgctctgttgccaggctggagtgcagtggagcgatcttggctcactgcaacctccgcctcccgggttcaagcaattcccctgcctcagcctcctgagtagctgggactacaggcgcccgccaccacgcccggctaatattttgtattttaatagagatggggtttcaccatgttggccaggatgttcttgatctcctgacctcatgatccgcccacctcggcctcccaaagtgttgggattgcaagtgtgagccaccgcgcctagaccatggtagttaattttaagtgttcaattcagtgaccttaagtgtgttcataatgttgtgcaaccatcaccatgttgtctaaccattagcactatctgttttgagaacttttttttatcatcccaaattagaattctgtacctgtcaaatagtccccagtaatcctccctcccccagcccctggtaatctgtagtctacttttcgtctttttgaatttgcctattttaggttcctcatataagtggaattatgtggtatttgtccttttgtgttggcttacttcatttagcataatgttttcaaggttcatctgtgttgtagcatgtatatacaggttgaagcatccgttatccaaaatggttgtgaccagaagtggtttggatttcagattttttttttggattttggaatattcatagatacttaactggttcagcatccctcgtccaaaaatccaaaatcagatggagctcagtggctcatgcttgtaatcccaacacgttgggtggccaaggcaggaggatcgcttgagcccaggagttcaaccagcctgagcaacacaagaccctatctctccaaaaaaaaaaaaaaaaaaaaaaagatgaaagaaaaaaaaatccaaaatcaaatgctccagtgagcatttccttttagcatcatgtcaggctctaaaagttacaggttttggagcattttggatttcagatttttggattaacctgcattaatgctcaacctatatgaaattttattcctttttatggctgaataatgttccactgtatgtatatactacattttgtttatccattcatctgttaacagacacttaagttatttccacattttgggtattataaatagtgctgctgcgaacattggtgtacatgtatctgtttgagtccctgtttttagttattttggttatatacctaggaatggaattgctgatcatatggtaattctgtgtttaactttttgaggaactaccactgttttccacaatggcatcaccattttacattcccaccagcaatgcacaaagatttcagtgtctgtatccttgctaacacttattttccattttttgagtttttttgttttgtttttttaataatagccaatcctaatgggtatgtggtagcatctcatggttttgattttattttcctgactattgatgatgttgagcatcttttcaggtgcttagtggccatttgtccgtcatctttggagcaggaacaatgtcttttcaagtcctttgcccatttttaaattgaattttttgttgttgagttgtatataacaccttttttgaagtaaaaggtgcactgtaataatccagactgtgtttctcccttctcaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattataggtgggtttaaattgaatataataagctgacattaaggagtaattatagtttttattttttgagtctttgctaatgccatgcatataatatttaataaaaatttttaaataatgtttatgaggtaggtaatatccctgttttataaatgaagttcttgggggattagagcagtggagtaacttgctccagactgcatcggtagtggtggtgctgggattgaaacctaggcctgtttgactccacagccttctgtactcttgactattctacaaaagcaagactttaaactttttagatacatcattaaaaaagaaaaccataaaaaagaatatgaaaagatgatttgagatggtgtcactttaacagtcttaaaagcaatcgtgtgtatagcatagaattgcttggattggataaacagtggcattatatattttaaaaaataaaagttttgaaagattgaagaatttgggcattacagttctcttaaatctgacaaagctgcataaaactattaaaataatcattattatactattttatattctatttctttgagggtttagttttccaaaaactacatattaagcaaatgaatcactcagtggctatgtcatataataacgagttagcctagttataagaagtttaacattttatttaagaacattgttacagcatgtttactgtatagtctagtaatagaggaaaagacatttgggtgggtggtagtggtagtatttttatagaggagttaccaaatttcagctctattatccaagtttacccagctaatggtgttcggaaccgggaatttgagccaattctgactctgttgtctgctctgctccttcttttgtgctgtgtctttgaaagtcacctaaaattgtgagggaatgtaatttcaccccaaatttagagtttatgcacttgttatattgaaaatgattaacatgtagaagggcttttaatggaataagtggtgtagtaacttcagtgttgcctacctagaaatcaaaatctttctagttgtccactttgttttttgaaaaagtaatatgaaaattatgttaatgctttaattcaggtttttgtaaaatattttttatctttacacatttaacatacgtttctaaaattatagtctgttatatagcactttgggtctagaatttttcagtagtttctgttttactattatgatctacctgcatattaacctattaggttatagttttactatacttctaggtatttgatcttttgagagagatacaaggtttctgtttaaaaaggtaaagaaacaaaataactagtagaagaaggaaggaaaatttggtgtagtggaaactaggaattacattgttttctttcagccaaattttatgacaaaagttgtggacaggttttgaaagatatttgtgttactaatgactgtgctataacttttttttctttcccagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacaggtaagtaacactgaaataaatacagatctgttttctgcaaaatcataactgttatgtcatttaatatatcagtttttctctcaattatgctatactaggaaataaaacaatatttagtaaatgtttttgtctcttgagagggcattgcttcttaatccagtgtccatggtactgcttttggctttggtttctttctacattgaaaatttctcttcaattctgagcacatgttaacatttagaattcaagaggtggggatttttttttcccatggttacatatatatatatatatatatatatatatatatatatatatatatatataaagaacagggcaacaaatttttgcgttttctatttcggtagtacttttaaaccattatgtcatgtttctaggttaaacgttgttgtatttgaagaattttactttggcagaatttttttgaggatgtgtttatttctggagaaaggtctcattaaagaaagacaatacccagaaagccaacagaaattctgttactcatttaatgcatttttctgacaaaaattattgccagagagaacctgaattttgtttcaaaaatcatctttgttttaaaaatgactttttcttcaggtaaaataaaataatttcagttgctattatttaacctgtttgtatgaagagtttaacatataggaaatgaatacataaagataggaaggaattaattgttatatgtagtcatatgtctcttaatgacagggatactttctaagaaatacattgttaggtgattttgtcattgtgcaaacatcatagaatatacttacacaaaccttggtagtataacctactatacacctgggatatgtagtatagtctcttgccccagggatacaaacctgtacagtatgtaactgtactaatgactataaggcaattgttaacacaatggtaagttttgtgtgtctaaacctacacttgggctaccctaagtttatatatttttttaaatttctgttcaataataaattaaccttactttactgtaactttttaaactttttaatttttcctaacattttgacttttgtaatacagcttaaaacacacattatacagctatacaaatttttctttccttatatctttattctgtaagcttttttccatatttaaaattttttgtttgtttttacttattaaacttttttgttaaaaactaagacatgcatgcacattaacctaggcctacacagggtcaggaccatcaatatcattgtcttccacttccacatcttgtcccactggaagatcttcaggggcagtaacacacgtggagctgtcatctcctataataacattgccttcttttggaatacctcctgaaggacctatccaaggctgtttatagttaacttttttttttttttttttttttttttagtaaataggaggagtacactataaaataacaatataggtgctataccattatacaactgacagtgcagtaggtttgtttacaccagcatcaccacaaacacgtgagcaatgtgtcgtactacagtgttaggatggctataacatcactaagcaataggaacttttaaactccattataatcttatgggaccactatcacatatgcaatctcctgtggaccaaaatgtcattatgtggtacatgactgtactaagaaattgatccatctatattccatcaatttgtttagggctttttctggttacatttacctgtgagcccagaaaaccagttttgtagaaattaacttctgtaatgctaggagttaaaaaaaattgctgaacaacttttacattgttaaacatttaaaaacaagcgttctagaagtttatcaaatttcataaaggtgcaaaaatgtaaatgtaaatcattatccagctaatatatatgttgtatttccctagtaggagagcatatgtacctcttcctagttatacaaatttgatatatagtaaagaaacagtaaattctacttcaagtcattttgggaggattaaaaactgaatttctctagtttgaccattgtacagatttatctggcaattttactaaaacctgatttataggttaaacttggtgtatatcatatatcactttactttagaggaattaagatttcacataaatccatttccaggttccaaagaccaggaagaggcttggtttttgtttttctttttactgtctttacagtctccttgacttttcttaggagagaaggtactgagaaaacatgattctaatatttattattttttcttccaacattttcttatgaaacattttcaaatacaaaattgagttttatttaaaacatttgcaaatatactacctagattctaccattgttgttttatatttgctttacttacaacttttaaaagatgctttttataccactgaacattttagcttacatttcacaaagaaaagaaaaaatttaagagactttgcataatgttttaaggggttgcagtaaagaagtgcttcttatattttcttatgcatacaaatcagctgggcttattaaaatccagattctaattcagaaggtttaggtggggaccgagtctgcatttctaacaaactcctaggtggtatttttcttggtacttggaccatactttgagtagaaaagcagtagaggacataaaaagagtcttgttagtcccactttgttgctgtccacttctcatttgataatatcctaaaatagctgtgtctcctttttggtggttgtatgattactacctcagaagtactaattgattcttgctatttgaccttaatactttaatataacacagcattcatatttgatcagaaaactatctggcttccttttataagagatttttaggttttatacagttttgtggccttgggtttttttgtttgatttgtttttttgaaggtatataatatgtaagtagataaacaaatttgatttgtagacatttttatgtggatcatctaattaaaaatggagggatacagtatgaaagaatacttgtacttcttaacagagcactcaacctttcttttacatcctgtttcactgatgttattatgtaatttatgttgctaaactataaattagatatttaatttctgttctttgatttccttttattattaaatggacttgttgatttgcctagaaattaatttgcctttcaaaagtcttattaatcttcctccgttgaaattaatttgatatttgcatgcttctggaagactttaaagagctattccgagtaactgtagagattataaaatgaaatatgggaattttaataaattttacatctccagttactggtgaaaatgtcaagtcctcctttctgcagagtattttgttactcatctgttattcagcttatttatttatttatttatttatttatttttctttctttcttgttttttttttttgagacggagtcttgctttgtcgcccaggctggagtacagtggtgggatcttggctcactgcaggctccgcctcccgggttcacaccattcttctgcctcagcctcccaagtagctgggactacaggcacccgccaccatgccttgctaaatttttgtatttttagtagagacgggtttcactgtgttagccaggatggtctcgatctcttgacctcgtgatccacctgcctcggcctcccaaagtgctgggattacaggcatgagccaccgcgcctggcccttatttgttttttaaacaaaattagtgtgcatatccttgttgtattttatcggcaagttgttttatgccctaacttttggggtcttgatcatgagcctaaaacacgtaaacacccaaaaagaattatattccggttaaaggaacaaaacattcatttagaagttctcatccatgtaaatcagaggctggcaaatattttctgtaaagggccaagatagtaaatgttttaggctttgagggccacaagtggtatctgttgcatttttttttaattatgaccctttaaaatgcaaaaatcgttgttagcttgtgcatagtataaaaataggctggccgcatgctgtggctcatgcctgtaatcccagaaatgaggtgggaagccgaggtgggcacaccacctgaggtcaggagttcgaggccagcctggccaacgtggttgaaaccccgtctctactaaaaatacaaaacttagccaggcgtggtggcgggtgcctgttatcctggctactcaaggggctgaggcagtagaattgcttgaacctgagaggcagaggctgtagtgagcccagatcaagccagtgcacaccagcctggacgaccgagcgagactctgtctcaaaaaaaaaaaaaaaaggctgtggctgcatttggtccattggctgtaatatgctgattcctaattctctgggtaactttagtgtttgattagctactagaagttaggttaaacttttgtattttacaggctaactttaataatcttaaagtaaaacttaacatagttcatggaaaggaaatagaaattttaccctagtactcttttttttttttttttttttttttgaggcagagtctccctctgtcacccaggctggagtgcagtggtgggatcttggctgattgcaacctcctcctcctgggttcaagcaattcttgtgcctcagcctcccgagcagctgggactacaggcacgcaccaccacacctgactgatttttgtatttttagtagagacagggtttcgccatgttggccaggctggtcttgaactcctggcatcaagtgatcctcccatctgagcctcccagtgtgctgggattacagacgtgagtcactgtgcctggtctctagtattttttttttttttgagacggtctcactgttgccaggctggagtgcagtggcgcgatcctggctcactgcaacctccgcttcccggattcaagcgattttcctgcctcagcctcctgagtagctgggactatgggtgcacaccaccacgcccagctaatttttgtatttttagtagagacggggtttcaccatgttggccaatatggtctcaatctcttgacctcgtgatctgcccgtctcggcctcccaaagtgctgggattacaggcgtgagccactgtgcccagctgtactttttaagataagaattgcagggtatatatttttaccaacttaataacttataattttaaaaagctaattacttggctagaatataatgcgttacatattctttacactcagttcagtccatatctgaaaggcaaatagaattattttctgctagtacattgtgtagtccctatgttcctagtgtataaggactgttacctagttcacatttatctgggttgttgacagattttcctggtccctttggacagtgcatggccatgttggcaaaagctgtcaaaattgaaacattgacaccatgagaattgtgtgttttccagtctgctaaaatcaaaagtgggagggttcagtaaggtgaataacagaagcagagttttcggggtatctgttactcctcattcggcttttctgctctctgggggtctcaatttaaatataatgtgaaaattagttttacgaacctaaaaatgttgagtgattcatttcctggttttgttgttaatttctagatatttaaattaattgttagaagaaccccgttaaagaatgctttgcaaaacaacctccttatgtgctatgtctctgtttaatagtagttgagtttgtgtacatgagatcaatattttgaactatagctttttatgagttaaaaattgacggaacagttactgtgcacttgctgtgcaccatggtagtctcccaagtagtggtttttctgcatttcaatagtacatgagataggctgtgggtggcaaggtttcttgagaaagtgagggatgcacagttgggttttagaatacatcttgttcctccatgcccttccccaccaaaaggctggtagtcttgcatttgtatatagttagggtatttgatgtgttgcttccttgacagagttttgcaagaatttgcagatttaacaggaacaaaaacttacttaaaacaaaatctcttagtaaaagcatagtctagcaagatttagaatgatactttggctaacagtactttctctatatggagtgctttgtttccatagcctcacaagtatgttttcagataatagttgagttgaaaatgttgtcaatctcttgattttaaaaaatttacatatttaaagttgtatacttttgttcctacgtattttcagttgttcttaaagtttaataagtgacatttgaaaatgagtatatgtgtataaaaacaaaagtaggctaggcacggtggctcatgcctataatcctagcactttgggaggctgaggcaggcggatcacaaggtcaggagtttgagaccagcctgggcaatatggtgaaacccccctctactaaaaatacaaaaattagctgggtgtggtggtgcatgcctgtagtcccagctactcaggaggctgaggcaggagaatcgcttgaacccggaggtggcggttgcagtgagccgagattgcaccactgcagtccagcctgggcggcagagcgagactccatctcaaaaaaaaaaaacaaaaaaagaaaaagttaaaaaaaaacaaaaaacccccacaaaatgagtatatgtggcaacaagtcctattctcaaaaaaattattgtgtgctagttaagagcttaatgagtagccagtcggtattaaatatctgtttcagctatattttatctttaaaaattatctacagattttggaatgtgaaaaactagtgttttgtttcataggtatatactgtaggcattttaaaaataagagccagtgccagtggtttacagtgtacacaaggataatgttctcatgttctcttgatgtcagtatgactttaaagcatattatcaagaaataactaagtctgaaaaactgtggtaaataactggtactctaaaacctaagtttcttattactaaaaataagaaatggtaaaagtcaccctgtgctgttaattatatgagccactgaggtcctgacactgaattcttggtggtggataataatctcttctttttaattattggcttccaattctctctgcattgctggaaacaaaaatcatatatttcactattggtggtggggatgctgtcactgaaaaagtagacacattcatattgattttagaaataagttaaaatcaaaatttgcttctgctaaattagtagaggaccaatactgtttttctccttcatagtatgttttggtacttctacattgacattataacttttttttttttaaacagaaatagaagtttacattcttagaaaatttatgaaaatatgagcttttacctggtttgtgtgtgtgcgtatatatatacacatatttttaaatttcttacattgattttcaaattgaaagagaaccatttgtgaaagtatcttaacagagctcatgctttacattttacatgctacaaagttattttagtgccttaaattatttatgttgcttattaatgaaaattttggatacataattttttcaagacaaaggtaaaaataataaaccctttccttctgaggattaatgataaatataaactttaaaacgattaaaaaaatttttttagagacagggtcttgctctgttgcccagactgaagtgcagtggtgcagtcatagctcaatgaagcctcaaactcctgggcccaggcaaccctcctgcctcagccttttgagtagctgggacttcaggctcatgccaacatgcctaatttatcttatttttagtagagatgaggtctcaaactcctggcatctcttgccctctcaaagtgctggtactacaggcattagtcaccacacctgacacttaaaatcttttatatacaggtgtaagtgggtatctaacttaaagtgccaacgaatgtagttgaaagtttgtagttggcttagctaactagttaactaaattgattccattaaaaataagataagactgctcttagaatataatgatttttgttattcgttaaatataaatatatcactggatagtatatgttaatgacttgagatacgcattttaacatataatcacgttacttaaatgcctgcctttgaactgaaacttaacattatgaatttaaattaaagtttgactttagaggtaaatttctgtactttactaaagcagttcttaatataattctgagatttctaaaaattagtgtgccctaaagaattgaggtgtgtttttcttaactactgtaggcagtagatgtacagatgacttctgcatgcaaaaattaagccctagccattggtttacttcaactaatacttagttgccaattctctgtgtgtgattgaatttaaaactgcaaatggtactggtgatacattaactttttaggtgctaggtccactttgttacatttggttcagtagaaacattgatgttaccaatctcagaaagctaaaatatgtatgccaatccccaaattaggtaatttattcttaattttaagataaaagaatagaattcccttaaaattaaatgtggagtaaaatataccagctttaaaaaatattcacctttctgttagaagaatgaacataatattacatcttttaatttgcactatatatagattaatatttctgtgtatttctctgtgcccctactttgatggtatgcttttctgaacaaactagcagcacagttaactaagcactttgccccgtttgatgactgcctaattttctagattggaaaatattaaaaacttttatctccatatggccaatatatgattgtacctgttgtcatagctctcttatgtttaagcaagaaaaaccctattaagagtatttaaattagaatggaaggcacacagccagtatgattgaacactgttctaaaaattatttttaagacttgtagtaaggccaggtttggtggctcatggctgtaatcccagcccttaggaggccaaggtgggcggatcacttgtgctcaggagtttgagaccagcccgggcaacatggcaaaaccctgtctctacgaaaaatacaaaaatcagtcaggtgtggtggtgcttgcctgtagtcccagctatttgagaggctgaggcagggggatcacctagcctgggaggtcgaggctgcagtcatgatcgtgccattgcactccatcctgggcaacccagtgagaccctgtctctaaaacaaaaaaataaaaaaagaacttgtagtaaggatacaaaatgctcctattttgtgtgtgtcctttaattcatgatgtttttatattatggtaagcagctctcatttaagattttaataatgtaattaaacatgtacagaagacccagtctcagcttcacttgtataccctggaaatagactgaaaggtgttaaaatttaagataaaactcaaggttccagtttcttgactcacctttgagattcttttatgtttttgttgttttttaacaaaggtttcacgtccatattttaccatttttcttctcattctcccctggaggagggtgtgggaatcgatagtatataaatcacttttttcctaagtcaaagaagtaatttaaagctaacttcagtttaggctttaattccaggactagcaaactaaaatggttgcattaattgacaaacagatgctaatacctgtgtttaggcttgtcataatctctcctaattcctaatttaaaaattttaaaatttaattccattagaaaacaaaactgacttttaagaacaaaccaggattctagcccatattttaaaactgcatcctcagttttattcaaacagtctgatgtctgtttaaaaaaaaaaaaatctcaagctcataatctcaaacttcttgcacatggctttcccagtaaattactcttaccaatgcaacagactttaaagaagttgtgttttacaatgcagagagtggaggatgctttttatacattggtgagggagatccgacaatacagattgaaaaaaatcagcaaagaagaaaagactcctggctgtgtgaaaattaaaaaatgcattataatgtaatctggtaagtttaagttcagcacattaattttggcagaaagcagatgtcttttaaaggtaacaaggtggcaaccactttagaactacttaggtgtagtattctaacttgaagtattaaaagataagaaacttgtttccataattagtacatttatttttaatctagtgggaattaattataattgagacaattttgatggctgtagtagactaatctatatttggcataaagtctaatgatttaatgagtcttaagtaaactaaatatttggaaactgatatttacctttatttttaagggaaaagttttgagataatcagcagcttttttttttttttttttttttttagtagggagaaaaagatatgagctatagtagacagcagtaatattgaatggcccagaaggtgggaaaaagccactcttaaatgtattttttcttttggatattttacaagcaaataataacttctgcctaagttcgccatctcagtggcatcagcagcacagcactttcttatcccagtgagaaacctgggaattttaggatgactcctaccgccctcttttccccctggtttggaagtatccacaaattcctgtgacgttacattctgtgtcttttatgtcatcattagttcaggcccctatcatttcttgttggactgttagaacctcctatttggtttaccagttgctgccatcattcattgtgaaaccggagagatacactttaaagaaatgtcatttttggccgggcgcggtggctcacgcctgtaatcccagcactttgggaggcctaggcgggtgatcacctgaggtcaggagttcaagaccagcctggctaacatggtgaaaccctatttctactaaaaatacaaaaaattagccgggcgtggtggcacgtgcctgtaatcccagctacttgggaggctgaggcaggagaattgcttgaacctgggaggcagaggttgcagtgagctgagaatgcaccattgcactccagcttgagcaacaagagcgaaactctgtctcaaaaaaaaaaaaaaaaagtcattttagctatagaataaaatctcatgttccacatgtgttgcagatagtccttactaccttcccaccactccagctcttttttggtcttatatctaaaaacgtcatcttgcctgaatttcttttgttcttctataaataaataccatgttatttcctaccttcccttgagtcttggctcttgtttggaatgccagtatttttatccctagtcttactaattagctaacactctcatgattccccagtctcctactctctaaaaacctttctttaaacccttagactaggcatggagcccttcctgtgtattcccagaatactattcttaactattatatgcttcccatgttatgttgaaataactaacctcttctgtttcattcctatattacttgacagcaaaatcttagccagaattacatatttttaatctttgcacacccattgcctagtaaggttcctgggacatagtaactacccagtaaatatttattgcgtggaattctcattttcgtttctaaacccgtattaaactctgtcttgctcagaaaatacttcactaggtatcataaagttcatggcagagcttaagctttggatgcatattgtttgtaatatatcatgttcttaagaataggcaataaaattacagttttcaaaaactactacatttattatatttattacaagttggtgttctttattacatgaattttaggtatttcccaaaagtataaaatatacatttgaatagtagactcaatcccaaaagatactacgtggtgtactaatctactaaactcagaaacaaagcatgactggcattaatttttgttgaaatttatgaactctgaatgtttttgaatatcattctgtaaagcaatattttgcaattaaagcaattttgcatgttaaattttaccacaacctctaaaatattgcaaatttaacaatacagtttgaaaagttacacattttaaataacagtaccatgaccagatttaggtggtggttttaattttttattttctcctcctattgtctcaccattagatgattttaaaaatagaattgtttagagtaaaataagtgttatgctctaatttatatttaaaatgaaggtttaagcacgtactattctaaaatttctaatttgtgcaaattatgttttatacagtgactgtaggtgaatgtcacaattgtttgatgtgacgaatccttgtttttcagtacacgtggaagtaattcatataaaagagaagtatacttggtaattaaaaatttaaaattaaatacaatttaaaaaaaaatttatttgacaagctggctgtggtgtgtgtgcctgtagtatcagctgcttgggagcctgaggcaggaggattgcctgaccccaggagtttgaggttgaagggagctatgatggtgccatggcactgtagcctaggcaacagaaagagactccatctcttaaaaaaagtaaaaataaaaaaattttggcacagggacagtggctcacacttataatgccagaactttaggagtccacagcgcgaggactgcttgaggccaggagtttaagaccagactgggcaacgtaatgagaccccacctttaggaaataaatacataaataaaaatttgacaatgataaacatatataaattagcttttcttagtcctgaaaaagataatgttatgtgtatgtgtgagaatgattagttctcatatgagaaaaaaagaattcattgctctgtgtaggttgtgacatttccttcacgattgaaattaattaatttttttttattacttatttatttttaaaatagagacaggttcttgctgtgttgcccaggctggtctcaaactcctggcctcaagcagttctcctgcctcagcctcccaaattgctgtgactgtaggtgtgagccactgcactgggccaaaattacttaattttaacaagatgatgtagagaggagagttcattgcaacataagcctagaatctttgtcagaatcttaggaagtaatgttttcaaattctgtgttttcaccataaaatgtgtcttctctgtgtccatcacatggtttttcattgttttctgctttaccattttagtaccattggcatttttcttcattgtaaaagtagtagaaatggagtagattacataaggatgtgatcagagggaatttattcattcagggtaagggagttagatcctcttttaagattctatcacattctaagggtttatgattctaaactgtcaagtaaattgtcaagtgctggcaagctacagaataatttttattgtatcattggaaattttcccctctatatgtgttaaagagtttagcctgaagggatacatacacatacatatatgtaatcaaaccttgatggtattgtattgctgataaattatttcttaccacttttcctttctcctgtgggagaaacaaaagcatatgtttgtgtagtatcagtaatgatattagagagtgggaaacatcagtgagtgcagtttggggactttattggagactttcactagtgctcaaataaataatgctggtttttatcctactgtttgcttaatgtggactagcctcttattcccattctatgtttacctctcttaaaatattggtcacgctttcttgaattatagatctattaggaaaattcatgaactgtagctaattttcattgttcatgctccagatttattttgaaatatcgttaatcttagtagtacagtaaaggagaaataccacttaacattttttgtttttttttctttgagacagagtcatgctctgtcacccagtctggagtgcagtggtgctatctcggctcactgcaatgcacttcgcctctccgggttcagcaattctcctgcctcagcctcctgagtagctgggattacaggcacctgctaccacacccagctaatttttgtatttttagtagagacagggtttcaccatgttggccaggctggtctgaaactcctcacctcaagtgatccacccgtcttggcctcccaaagtgctgggattacaggcttgagccaccgcaccccgcccacttaacattttaaattaatttcaagataatatcacttgaatatttttacacatataatttttttaatacatttatttacacagtttataatatcctacaaagtgattacaatgagtaaaaacccagttttcattgttcctaaagtggcttgatttatacaacttaatgtgttgggtatttgtttctaagactccctctgctgtctaggtttggaagtattgtgaggttaacagattttctttttatagttactactcagttgaacaggctttaaaatacagagagaatcatattttttcttcattttttgcttttatttatatttttcttttaattggagacatgacaagaattgacttgtgtatggatcttgcataatttaagtactgcaggtttaaaatctactaccagtttgagagtgccatttttcacactgtagattattaggttgaaaagtattatggcttaaaatcgcttttagccattaaatttaaataaccttgctttaatcataaatagatggtggtcacaatgactaactgttaaactctttgaagacaggatatttggctttatatggcaagcttttgaatacaacagaaattaaaactttatgggatagaaagaatctcctccaaattggtaaactataagacctttcaaatgatttagctaatttctccacaaatctgaggtattagtgttttttttaaagtggtattctcctgtgttggggtcactttaaacctttttcttaatgataaatatatgaattgaaactaatcccttaatatatatcatttgaaaactgaaataatatgtttagatactgtttacttgttgataaattattggaataggatgttcgaatactgtttacttcttggtaaatttttaaatccaatggattttacgtaagtatagaactggagctcaaatactgttactgtgtgtgaagatatatgaacatagtttacagttgcatggcttatatctaaagtccagaaacataaggacaattaagtgtacacacacacacatgcatttggattttgatgacttaggtttgccaatgtggaaaaaatagtagcaaattaagttctcctgtgaaaaagtcgttaccttatttaaaattctgtgccattggttatccttgtcttttgtgaaaattagtgttcctgtttataatattgacaaaacacctatgcggatgacatttaagaattctaaaagtcctaatatatgtaatatatattcagttgcctgaagagaaacataaagaatcctttcttaatattttttccattaatgaaatttgttacctgtacacatgaagccatcgtatatattcacattttaatactttttatgtatttcagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccaggaactgtttgttttgtagtgaaccttgagtatgtgctgttaatataccaaattgggtgaaaaaataagggattcctttcaaaagttaagagaagtaagtgtgtaagaaattattttgcttattaaatgttcggtaaatggcattctcttgtcagtaaaatggagaaataagctaaaaataattggctaagtcctattaagttagaggattaagtgtattatattttcattcaaaattgggtgctcattaatttatgatcggtagtatagctaaattgctatgtttgtatcaaaattgagcataaagttgctgatactttctccgtatgaacagaagttgaaacctatttagttcagtagggcagctcagggattttttacacaacatgtatatcttcccattttaagttagaattattttacaacatctggtatacataaacagctggcactgatagctaaattaaagtagtaatgatcaattagttttgttggtatctgaataatagcgttgtttcatagctctgtatttcctaaggaagtacaaagcttctagctctttcattacaaattcgccctgtgcaataagttctttgatcttctctggattcttcacatctttgtttttaaggaaaatgttcttcaaacgctttttaaaatagtctgctccttttggatagtctcgtccaagatacagcagcttcaaaaagaaagattatatatttctaaacaatccatgtcatataataacatttttataaaattggcaacataattacttacatttttataaagttttagtacttctcctcttaaagaattggccattttcatttatcatgtaaattatccacttttatgcataacatacctaaagaaaggaaaatttttttgcaattagctgcattgtagtcttaaaaaaataaaaaaaggttatacacattgagaaaatggtaaccttttttacattcaataaatatttcttgataactttttcgttccacgtactgggatatagttataaacacttccgataaaattacctgctgtcataattgacgttttcctatgggagacataagcaaagacaattgtgattgtgagaagtcacatgaaggaaatgagaaagtggattgtcatcacagataggtacgtgtacctccttttatgccacagtggaatgagttaaactagatttaaattccagttgcataatgtacagattaattaaccttgctgagcctgagttttccttatcaacaaacaagagattatctttaccctgctctcaaggcaaggccagagccacttgaaggacattgagcagaagcctgatcaaatgctgatgggtgcttatccaaagggaggctgaaaactagcagaaactgggtgagttaagcaggttggaatagtagatgggcagtaagattggtggtgaagaggccaaatgaacaacctgtaagagggtgtccctgaggaacaggcaaaatcatgcttctttatgtgtaatgtgttaactctactttgtagaggaggctccaaact

SEQ ID NO: 13 - DNA, 2513 bp, Homo sapiens
cgcctcccggccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtggaaaaaaaaaaaaaaaaaaaaaaaaaaaaaaaaaaaaaaaccc

SEQ ID NO: 14 - DNA, 3724 bp, Homo sapiens
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa

SEQ ID NO: 15 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa

SEQ ID NO: 16 - protein, 189 aa, Homo sapiens [sequence omitted]

SEQ ID NO: 17 - protein, 766 aa, Homo sapiens [sequence omitted]

SEQ ID NO: 18 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 19 - protein, 403 aa, Homo sapiens [sequence omitted]